(19) United States
(12) Patent Application Publication
(10) Pub. No.: US 2021/0407677 A1
(43) Pub. Date: Dec. 30, 2021

(54) FATTY ACID AND KREBS CYCLE METABOLOMIC MEASURES FOR TREATMENT OF HEAD INJURY

(71) Applicants: Northwestern University, Evanston, IL (US); The Penn State Research Foundation, University Park, PA (US)

(72) Inventors: Hans C. BREITER, Evanston, IL (US); Semyon SLOBOUNOV, University Park, PA (US); Nicole VIKE, Evanston, IL (US); Sumra BARI, Evanston, IL (US)

(73) Assignees: Northwestern University, Evanston, IL (US); The Penn State Research Foundation, University Park, PA (US)

(21) Appl. No.: 17/352,045

(22) Filed: Jun. 18, 2021

Related U.S. Application Data
(60) Provisional application No. 63/041,606, filed on Jun. 19, 2020.

(51) Int. Cl. G16H 50/20; G16B 25/10; G16B 20/20; G16H 50/70; G16H 10/60; G16H 10/40

(52) U.S. Cl. CPC G16H 50/20; G16B 25/10; G16H 10/40; G16H 50/70; G16H 10/60; G16B 20/20

(57) ABSTRACT

Methods relating to treatment and diagnosis of head injury are provided herein.

### CROSS-REFERENCE TO RELATED APPLICATIONS

This application claims priority to U.S. Provisional application No. 63/041,606, filed Jun. 19, 2020, the entire contents of which are incorporated herein by reference.

### FIELD

The present disclosure relates to medical methods for treatment and diagnosis of head injury or concussion.

### BACKGROUND

Given the heightened awareness of concussive and sub concussive-related morbidity, large efforts have been made to elucidate potential biomarkers of brain injury [1-8]. While many promising biomarkers have been discovered, they often lack a relationship to neurological function or dysfunction (observed with behavioral testing or imaging) and other markers of neuroinflammation (i.e. miRNAs) across heterogenous populations [9-11]. Ultimately, a biomarker, or set of biomarkers, that show a relationship to neurological function and neuroinflammatory miRNAs could be used to 1) detect and predict neurological dysfunction and 2) assess the efficacy of novel brain injury interventions.

Metabolomics proves as a viable method to investigate metabolic fluctuations following a particular exposure, such as contact sport participation. Many molecular pathways have been studied in animal models and have been observed to be dysfunctional [12-19]. In particular, mitochondrial dysfunction has been consistently observed following concussive and subconcussive-related brain injury [20-26]. Dubbed the powerhouse of the cell, mitochondria are critically important for energy production and cellular respiration [27, 28]. Damage to these organelles can thus result in serious cellular, and potentially systems-level, dysfunction.

One major role of the mitochondria is the oxidation of fatty acids into functional metabolites that can then fuel the TCA cycle and downstream energy production [29-31]. Long and medium chain fatty acids are first processed in peroxisomes, small organelles that oxidize fatty acids via alpha, omega, and beta oxidation [32-34]. Omega oxidation replaces the methyl terminus with a hydroxy group (now referred to as a monohydroxy fatty acid) which is then further oxidized into a carboxy group (dicarboxylic fatty acid). Once in dicarboxylic form, the fatty acid can then be further processed via beta oxidation in both the peroxisome and mitochondria [35, 36]. Depending on the length and processing location of the fatty acid, different sets of enzymes are utilized to metabolize it into a smaller, functional metabolite-acetyl-CoA. Acetyl-CoA then enters the TCA cycle to produce energy (GTP) and energy-storing (NADH and FADH₂) metabolites [37]. NADH and FADH₂ are then used in the electron transport chain to ultimately produce large amounts of ATP—the most fundamental source of energy. However, if mitochondrial damage occurs from repetitive head impacts (i.e. head acceleration events, HAEs), metabolites involved in fatty acid oxidation (e.g. sebacate) and the TCA cycle (e.g. citrate) may be altered [21]. Additionally, mitochondrial distress can result in oxidative stress and increased reactive oxygen species production [38-41]. Therefore, finding a set of metabolites to highlight potential mitochondrial dysfunction, while also showing relationships to neurological function/dysfunction and neuroinflammatory miRNAs, is a promising and novel biomarker.

Neurological function can be assessed using many different techniques: neuroimaging (MRI, CT, PET), standard behavioral tests (e.g. PCSS and GCS), and computational behavioral tasks (e.g. virtual reality) [42]. While PCSS is commonly used when concussion is suspected, it is not sensitive enough to detect subconcussive injury [43]. On the other hand, various magnetic resonance imaging (MM) techniques (e.g. rs-fMRT, fMRT, DTI, and MRS) have been shown to reveal subtle changes in neurophysiology, such as decreased or increased blood oxygenation [44-49]. However, imaging is costly and not suitable as a subconcussive biomarker. Behavioral testing offers itself as an inexpensive way to diagnose dysfunction, but observable behavioral changes are rarely observed, especially when investigating subconcussive injuries [48, 50].

Therefore, there exists a need for reliable and practical method to identify concussive head injury and biological indicators of the same. The present disclosure satisfies this need.

### SUMMARY

One embodiment is a method of treating head injury, diagnosing head injury, prognosing head injury, assessing susceptibility for head injury, assessing resilience against head injury, and/or determining treatment for head injury, in a subject comprising: comparing, using a computer processor, at least three measures selected from the group consisting of: a) fatty acids and metabolites thereof; b) TCA metabolites; c) nucleosides, nucleic acids, and derivatives thereof; d) behavioral scores; and e) imaging scores, from the subject, to the same measures in a control subject.

Another embodiment is a method of identifying a biological target for a therapeutic agent to treat or prevent head injury, the method comprising comparing, using a computer processor, at least three measures selected from the group consisting of: a) fatty acids and metabolites thereof; b) TCA metabolites; c) nucleosides, nucleic acids, and derivatives thereof; d) behavioral scores; and e) imaging scores, from a subject, to the same measures in a control subject.

Another embodiment is a method for detecting at least two biomarkers of head injury in a subject, the biomarkers selected from the group consisting of: a) fatty acids and metabolites thereof; b) TCA metabolites; and c) nucleosides, nucleic acids, and derivatives thereof, the method comprising contacting a biological sample from the subject with an agent specific for the biomarker(s) and detecting the presence or absence of the biomarker in the biological sample based on the agent's interaction with the biomarker to determine a head injury biomarker profile for the subject and comparing the subject's head injury biomarker profile to a control head injury biomarker profile.

### BRIEF DESCRIPTION OF THE DRAWINGS

FIG. 1 shows a random forest plot of metabolite importance. The mean decrease accuracy (MDA) is a measure of model performance when a particular metabolite was excluded from the analysis. As MDA increased, the more important that metabolite was to distinguish pre-versus post-season data. The metabolite with the highest MDA, and therefore, highest predictive power, was 2-hydroxyglutarate, a fatty acid. The majority of metabolites in the plot were lipids (55%).

FIG. 2 shows significant preseason mediation results. In all analyses, miRNA was the independent variable, metabolite was the mediator, and VR score was the dependent variable. Cook's distance outliers were removed prior to analyses, VR terms were reported as standardized values, adjusted R² (R_{adj}²) values were reported for each regression, T_{eff} represents the percentage of the total effect mediated by the metabolite, and all p-values were reported at a significance level 0.05. The graph depicts the change in slope between model 1, which plots the slope term for the interaction between miRNA and VR score, and model 2, which plots the slope term for the interaction between miRNA and VR score when metabolite was included in the regression model.

FIG. 3A-3C shows significant preseason mediation results. In all analyses, ΔmiRNA was the independent variable, Δmetabolite was the mediator, and ΔVR score was the dependent variable. Cook's distance outliers were removed prior to analyses, ΔVR terms were reported as standardized values, adjusted R² (R_{adj}²) values were reported for each regression, T_{eff} represents the percentage of the total effect mediated by the metabolite, and all p-values were reported at a significance level 0.05. The graph depicts the change in slope between model 1, which plots the slope term for the interaction between ΔmiRNA and ΔVR score, and model 2, which plots the slope term for the interaction between ΔmiRNA and ΔVR score when Δmetabolite was included in the regression model.

FIG. 3D-3F shows significant preseason mediation results. In all analyses, ΔmiRNA was the independent variable, Δmetabolite was the mediator, and ΔVR score was the dependent variable. Cook's distance outliers were removed prior to analyses, ΔVR terms were reported as standardized values, adjusted R² (R_{adj}²) values were reported for each regression, T_{eff} represents the percentage of the total effect mediated by the metabolite, and all p-values were reported at a significance level 0.05. The graph depicts the change in slope between model 1, which plots the slope term for the interaction between ΔmiRNA and ΔVR score, and model 2, which plots the slope term for the interaction between ΔmiRNA and ΔVR score when Δmetabolite was included in the regression model.

FIG. 3G-3H shows significant preseason mediation results. In all analyses, ΔmiRNA was the independent variable, Δmetabolite was the mediator, and ΔVR score was the dependent variable. Cook's distance outliers were removed prior to analyses, ΔVR terms were reported as standardized values, adjusted R² (R_{adj}²) values were reported for each regression, T_{eff} represents the percentage of the total effect mediated by the metabolite, and all p-values were reported at a significance level 0.05. The graph depicts the change in slope between model 1, which plots the slope term for the interaction between ΔmiRNA and ΔVR score, and model 2, which plots the slope term for the interaction between ΔmiRNA and ΔVR score when Δmetabolite was included in the regression model.

FIG. 4A-4C shows significant moderation analysis result. (A) ΔVR Balance was the dependent variable, DMN fingerprint and Δtridecenedioate were independent variable and moderator (p_{F}^{perm}=0.05, p_{B}_{₃}^{perm}=0.03). The regression slope (β), p-value and adjusted R² (R_{adj}²) depicted on each arm of the triangle corresponds to the significant interaction results, after Cook's distance outliers removed, between those two variables with p-values reported at a significance level of 0.05. (B) Interaction plots corresponding to the moderation analysis. There was negative relationship between DMN fingerprint and Δtridecenedioate; there was negative relationship between Δtridecenedioate and ΔVR Balance; there was positive relationship between DMN fingerprint and ΔVR Balance. (C) The plot depicts the relationship between DMN fingerprint and ΔVR Balance with Δtridecenedioate as the moderator. Three lines corresponds to low (mean−standard deviation), medium (mean) and high (mean+standard deviation) Δtridecenedioate values.

FIG. 4D-4F shows significant moderation analysis result (D) DMN fingerprint was the dependent variable, and ΔmiR-505 and Δtridecenedioate were independent variable and moderator (p_{F}^{perm}=0.02, p_{B}_{₃}^{perm}=0.02). The regression slope (β), p-value and adjusted R² (R_{adj}²) depicted arm of the triangle corresponds to the significant interaction results, after Cook's distance outliers removed, between those two variables with p-values reported at a significance level of 0.05. (E) Interaction plots corresponding to the moderation analysis. There was positive relationship between Δtridecenedioate and ΔmiR-505; there was negative relationship between Δtridecenedioate and DMN fingerprint; there was negative relationship between DMN fingerprint and ΔmiR-505. (F) The plot depicts the relationship between DMN fingerprint and ΔmiR-505 with Δtridecenedioate as the moderator. Three lines corresponds to low (mean−standard deviation), medium (mean) and high (mean+standard deviation) Δtridecenedioate values.

FIG. 5A-5C combines the moderation analyses results with HAEs. (FIG. 5A) ΔVR Balance was the dependent variables and ΔmiR-505 was the dependent variable. DMN fingerprint acted as independent variable for one moderation and as dependent variable for the other one. Δtridecenedioate acted as the moderator for the two analyses. The regression slope (β), p-value and adjusted R² (R_{adj}²) depicted arm of the diamond corresponds to the significant interaction results, after Cook's distance outliers removed, between those two variables with p-values reported at a significance level of 0.05. This figure depicts the integration of resting state fMRI with metabolic profiling, transcriptomics and computational virtual reality (VR) behavior task along with Head Acceleration Events (HAEs). (FIG. 5B) Interaction plots between Network fingerprint, ΔmiRNA with Head Acceleration Events (HAEs). The regression slope (β), p-values and correlation coefficient (r) depicted on top of the plots corresponds to the interaction results, after Cook's distance outliers removed, between two variables with p-values reported at a significance level of 0.05. There was a positive relationship between ΔmiR-505 and average number of HAEs above 25 G per session (aHAE₂₅₆). (FIG. 5C) There was a negative relationship between DMN fingerprint and cumulative number of HAEs above 80 G (cHAE_{80G}).

FIG. 6 summarizes the observed shift in metabolism. There were significant increases in medium-chain monohydroxy and dicarboxylic fatty acids (FAs) (suberate, sebacate, UND, and 8-HOA) from pre- to post-season. Increases in these FAs have been associated with genetic disorders related to impaired beta-oxidation. Impairment of this critical process can result in an accumulation of medium-chain FAs which cannot be further oxidized into smaller, functional, metabolites, such as acetyl-CoA. Acetyl-CoA is a critical starting-point metabolite for the TCA cycle, a major source of energy-rich molecules that are fed into further energy-producing processes (e.g., electron chain transport system). Here, TCA-related metabolites (citrate, aconitate, α-KG, fumarate, and malate) were all observed to decrease, suggesting a problem with the initial step of the cycle (i.e. lack of acetyl-CoA). Additionally, there were alterations in energy-rich molecules such as adenine, adenosine, nicotinamide, and phosphate, suggesting a state of energy imbalance. Lastly, 2-HG, a known oncometabolite, was observed to increase. Regardless of its role as an oncometabolite, its increase suggested a state of oxidative stress. Taken together, it is suggested that there are dysfunctional beta-oxidative processes in this cohort of collegiate football players leading to subsequent issues with energy production.

FIG. 7 summarizes the observed shift in metabolism and its relationship to inflammatory miRNAs and complex behavior. Repetitive exposure to subconcussive head accelerate events (HAEs) has been shown to produce significant changes in brain homeostasis, such as increased neuroinflammation (cite). Here, we observed changes indicative of mitochondrial distress as evidenced from both the accumulation of medium-chain FAs and the subsequent decreases in TCA-related metabolites. Mitochondrial dysfunction can lead to numerous physiological disturbances, some of which were observed in the present study: 1) oxidative stress (i.e., increased levels of 2-HG), 2) impairment in beta-oxidative processes (i.e., increased levels of medium-chain FAs), and 3) increased metabolic demands (i.e., decreased TCA and energy-related metabolites). Together, these neuroinflammatory processes may be related to the observed elevation in inflammatory-related miRNA molecules (specifically miR-20a, miR-505, miR-151-5p, miR-30d, miR-92a, and miR-195). In fact, these miRNAs were significantly correlated with the metabolites shown in 3A. In addition, the metabolites were shown to mediate the relationship between elevated miRNA levels and complex Luria behavior (i.e., computation virtual reality tasks). Together, the mediating effect of these energy-related metabolites are critical when defining the relationship between elevated miRNAs and behavioral outcomes. This complex relationship may explain why obvious behavioral changes in subconcussed athletes have not been routinely observed, but how repetitive, long-term exposure to HAEs, chronic elevation of inflammatory-miRNAs, and acute, but deleterious changes in energy metabolites could result in behavioral disturbances later in life.

FIG. 8 summarized the observed metabolic disturbances. There were significant increases in medium-chain monohydroxy and dicarboxylic fatty acids (FAs) (suberate, sebacate, UND, and 8-HOA) from Pre to Post. Increases in these FAs are associated with genetic disorders related to impaired β-oxidation, which can result in an accumulation of medium-chain FAs that cannot be further oxidized into smaller, functional, metabolites, such as acetyl-CoA. Acetyl-CoA is a critical input for the TCA cycle—a major source of energy-rich molecules that are fed into further energy-producing processes (e.g., electron chain transport system). Here, TCA-related metabolites (citrate, aconitate, α-KG, fumarate, and malate) all decreased, suggesting a problem with the initial step of the cycle (i.e., lack of acetyl-CoA). Additionally, there were alterations in energy-rich molecules such as adenine, adenosine, nicotinamide, and phosphate, suggesting a state of energy imbalance. Lastly, 2-HG increased. Regardless of its role as a known oncometabolite, its increase suggests a state of oxidative stress. Together, it is suggested that there are dysfunctional β-oxidative mitochondrial processes in this cohort of collegiate football athletes leading to subsequent issues with energy production.

### DETAILED DESCRIPTION

As used herein and in the claims, the singular forms “a,” “an,” and “the” include the plural reference unless the context clearly indicates otherwise. Throughout this specification, unless otherwise indicated, “comprise,” “comprises” and “comprising” are used inclusively rather than exclusively. The term “or” is inclusive unless modified, for example, by “either.” Thus, unless context or an express statement indicates otherwise, the word “or” means any one member of a particular list and also includes any combination of members of that list. Other than in the examples, or where otherwise indicated, all numbers expressing quantities of ingredients or reaction conditions used herein should be understood as modified in all instances by the term “about.”

Headings are provided for convenience only and are not to be construed to limit the invention in any way. Unless defined otherwise, all technical and scientific terms used herein have the same meaning as those commonly understood to one of ordinary skill in the art. The terminology used herein is for the purpose of describing particular embodiments only and is not intended to limit the scope of the present invention, which is defined solely by the claims. In order that the present disclosure can be more readily understood, certain terms are first defined. Additional definitions All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art and are set forth throughout the detailed description.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention. When an embodiment is defined by one of these terms (e.g., “comprising”) it should be understood that this disclosure also includes alternative embodiments, such as “consisting essentially of” and “consisting of” for said embodiment.

“Substantially” or “essentially” means nearly totally or completely, for instance, 95%, 96%, 97%, 98%, 99%, or greater of some given quantity.

The terms “subject,” “individual” or “patient” are used interchangeably herein and refer to a vertebrate, preferably a mammal. Mammals include, but are not limited to, mice, rodents, rats, simians, humans, farm animals, dogs, cats, sport animals, and pets.

The term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term. For example, in some embodiments, it will mean plus or minus 5% of the particular term. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the disclosure.

As used herein, the term “treatment” or “treating” means any treatment of a disease or condition or associated disorder, in a patient, including:

Inhibiting or preventing the disease or condition, that is, arresting or suppressing the development of clinical symptoms, such as neurological deficits resulting from cerebral ischemia, also included within “treatment” is provision of neuroprotection; and/or relieving the disease or condition that is, causing the regression of clinical symptoms, e.g., increasing neurological performance or reducing neurological deficits.

In some embodiments, “treatment” encompasses “providing neuroprotection” to the subject. “Treatment” and “providing neuroprotection” may comprise the administration of the therapeutics agent(s) or compositions disclosed herein.

“Pharmaceutically acceptable salt” refers to salts of a compound, which salts are suitable for pharmaceutical use and are derived from a variety of organic and inorganic counter ions well known in the art. Pharmaceutically acceptable salts include, when the compound contains an acidic functionality, by way of example only, sodium, potassium, calcium, magnesium, ammonium, and tetraalkylammonium. When the molecule contains a basic functionality, salts of organic or inorganic acids, such as hydrochloride, hydrobromide, tartrate, mesylate, acetate, maleate, and oxalate. Stahl and Wermuth, eds., “Handbook of Pharmaceutically Acceptable Salts,” (2002), Verlag Helvetica Chimica Acta, Zurich, Switzerland), which is hereby incorporated by reference for its teachings related to pharmaceutically acceptable salts, discusses a variety of pharmaceutical salts, their selection, preparation, and use.

“Prognosing” may refer to ascertaining the likely course of (severity, length of time, etc.) a disease or ailment, i.e., head injury. The severity of a head injury may be ascertained as measured by the techniques described below.

“Susceptibility” towards head injuries refers to the state or fact of being likely or liable to be influenced or harmed by the injury.

“Resiliency” against a head injury refers to the capacity to recover quickly from difficulties. Recovery may be quantified or qualified according to any of the methods, imaging techniques, or behavioral tests described below.

“Biological targets” refer to enzymes or metabolic/catabolic steps in a biological pathway that may be interrupted, inhibited, agonized, antagonized, or activated by a therapeutic agent or compound.

“Diagnosing” may refer to identify the nature of (an illness or other problem, i.e., head injury) by examination of the symptoms.

“Computer readable medium” is a medium capable of storing data in a format readable by a mechanical device (rather than human readable). Examples of machine-readable media include magnetic media such as magnetic disks, cards, tapes, and drums, punched cards and paper tapes, optical discs, barcodes and magnetic ink characters. Common machine-readable technologies include magnetic recording, processing waveforms, and barcodes. Optical character recognition (OCR) can be used to enable machines to read information available to humans. Any information retrievable by any form of energy can be machine-readable.

The present disclosure is directed to a method of treating head injury, diagnosing head injury, prognosing head injury, assessing susceptibility for head injury, assessing resilience against head injury, and/or determining treatment for head injury, in a subject.

“Head injuries” are defined as damage to the brain resulting from external mechanical force, such as rapid acceleration or deceleration, impact, blast waves, or penetration by a projectile. Brain function is temporarily or permanently impaired and structural damage may or may not be detectable with current technology. The injuries may include traumatic brain injury. Injuries can be classified into mild, moderate, and severe categories. The Glasgow Coma Scale (GCS), the most commonly used system for classifying TBI severity, grades a person's level of consciousness on a scale of 3-15 based on verbal, motor, and eye-opening reactions to stimuli. In general, it is agreed that a TBI with a GCS of 13 or above is mild, 9-12 is moderate, and 8 or below is severe. Similar systems exist for young children. However, the GCS grading system has limited ability to predict outcomes. Because of this, other classification systems such as the one shown in the table are also used to help determine severity. A current model developed by the Department of Defense and Department of Veterans Affairs uses all three criteria of GCS after resuscitation, duration of post-traumatic amnesia (PTA), and loss of consciousness (LOC).

The severity may be asses according to Table A:

TABLE A

| | GCS | PTA | LOC |
|---|---|---|---|
| Mild | 13-15 | <1 day | 0-30 minutes |
| Moderate | 9-12 | >1 to <7 days | >30 min to <24 hours |
| Severe | 3-8 | >7 days | >24 hours |

The method of the invention include comparing, using a computer readable medium, three measures selected from the group of:
a) fatty acids and metabolites thereof
b) TCA metabolites;
c) nucleosides, nucleic acids, and derivatives thereof;
d) behavioral scores; and
e) imaging scores,
from a subject, to the same measures in a control subject.

Fatty acids and metabolites thereof may include any fatty acid or metabolite known to exist in mammals or other organisms. Examples include tridecenedioate, 2-hydroxyglutarate, undecanedioate, sebacate, suberate, and heptanoate, or a conjugate acid thereof.

Nucleosides, nucleic acids, and derivatives thereof may include any polynucleotide known to exist in biological organisms. Examples include DNA, RNA and all subtypes of either thereof, i.e., sRNA, mRNA, siRNA, miRNA, and sRNA.

A TCA metabolite refers to a metabolite present in, resulting from, or used by the citric acid cycle otherwise known as “the Krebs's cycle.” Examples include adenine, nicotinamide, adenosine, acetyl-CoA, citrate, aconitate, isocitrate, NAD+, NADH, CO₂, α-keto glutarate, L-2-hydroxyglutrate, D-2-hydroxyglutrate, GTP, GDP, phosphate, pyrophosphate, S-CoA, coenzyme A (CoA), succinate, FAD, FADH₂, ATP, ADP, AMP, fumarate, malate, oxaloacetate, and conjugate acids any thereof.

Behavioral scores may be assessed using any of the scales described above for measurement of head injuries or virtual reality testing (VR) as described in the examples, Post Concussion Symptom Scale (PCSS), Glasgow Coma Scale (GCS), ear opening and eye opening, surface righting, air righting, forelimb grasp, auditory startle, surface righting, negative geotaxis, openfield traversal, cliff aversion, Barnes maze, elevated plus maze, Jamar dynamometer, handheld dynamometry, manual muscle testing (MMT), isokinetic dynamometry, trunk stability test (TST), unilateral hip bridge endurance test (UHBE), pronator sign, Barré sign, Romberg test, Landau reflex, particle suspension, sensory reflex (pinprick, light touch, position, vibration, and charger), reflex (biceps, triceps, brachioradialis, patellar, and ankle), Moro reflex, tonic neck response, sucking reflex, palmer and planter grasp reflex, parachute response, neck on body righting reaction (NOB), body on body righting reaction (BOB), ear opening auditory reflex, static compliance, physical volume of ear canal, contralateral reflex, ipsilateral reflex, tympanometry, Y-maze, Novel Object Recognition Task, STPI (State-Trait Personality Inventory), the Five Dimensional Curiosity Scale, Self-Curiosity Attitude Interests Scale, Curiosity and Exploration Inventory-II, State-Trait Personality Inventory (STPI), subscales of the Sensation Seeking Scale (SSS), Bayley Scales of Infant Development (BSID-III) (1-42 months), the Mullen Scales of Early Learning (1-68 months), the Fagan Test of Infant Intelligence (FTII) (Birth-12 months), Griffith's Mental Development Scales I (0-2 years), Battelle Developmental Inventory (BDI) (Birth-8 years), and the Vineland Adaptive Behavior Scale (0-18 years) or cognition tests including ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, and a computerized tested selected from Cantab Mobile, Cognigram, Cognivue, Cognision, or Automated Neuropsychological Assessment Metrics Cognitive Performance Test (CPT).

Embodiments described herein are further illustrated by, though in no way limited to, the following working examples.

### EXAMPLES

### Example 1: Analysis of Biological Markers Related to Head Injury in American Football Players

Purpose: Imaging and behavior are critical for the initial investigation of blood biomarkers linked to neurological dysfunction. The goal of this example was to establish a set of blood serum metabolites that had significant and complex relationships with computational behavior performance, advanced brain imaging, and inflammatory miRNAs. Once a set of metabolites was identified to exhibit significant relationships with behavior and imaging, they could then be used as a standalone biomarker for neurological dysfunction in the absence of observable symptoms related to repetitive head impacts.

Summary: This example identified a set of eight metabolites with significant mediating and moderating effects: tridecenedioate, 2-hydroxyglutarate, undecanedioate, sebacate, suberate, heptanoate, and adenosine. Additionally, six metabolites in biochemical relation with the aforementioned metabolites were observed to change across the season of football participation: citrate, aconitate, alpha-ketoglutarate, fumarate, phosphate, and adenine. Together, these changes indicate a state of mitochondrial dysfunction. In particular, there is an impairment of fatty acid beta-oxidation and a subsequent decrease in TCA cycle metabolites. This set of metabolites serve as an important biomarker for mitochondrial distress resulting from repetitive head acceleration events (HAEs), which has applications for diagnosis of concussion/head injury, prognosis of longitudinal course after head injury, assessment of potential susceptibility and/or resilience to head injury, prediction of potential treatment for head injury and development of biopharmaceutical interventions for head injury through intervention before, after or along the metabolomic pathways described herein.

### Materials and Methods

Subjects and data collection: Twenty-four male collegiate American football players (mean age=21±2 years) were recruited for this study. Written informed consent was obtained from each subject in accordance with the Penn State University Institutional Review Board. Demographic information was obtained from each subject and confirmed by a team physician: age, years of play experience (YoE), player position, and history of diagnosed concussion (HoC; reported as the number of previous concussions). Blood samples were taken prior to any contact practices (Pre-season) and within one week of the last game (Post-season). None of the subjects had a diagnosed concussion in the 9 months preceding preseason data collection. Blood samples were prepared and sent out for miRNA quantification and metabolomic analysis. Concurrent with blood collection, players were subject to virtual reality (VR) testing and an MRI protocol.

Serum Extraction: Five mL of blood were drawn from each participant at Pre and Post sessions. Samples were placed in a serum separator tube, allowed to clot at room temperature, and then centrifuged. Serum was extracted from each tube and pipetted into bar-coded aliquot tubes. Serum samples were stored at −70° C. until they were transported to 1) a central laboratory for blinded miRNA batch analysis and 2) Metabolon (Morrisville, N.C., USA) for blinded metabolite analysis.

miRNA quantification: Serum samples collected at Pre and Post sessions were used to isolate and quantify levels of RNA. 100 μL of serum was aliquoted and RNA was isolated using a serum/plasma isolation kit (Qiagen Inc., Venlo, Netherlands) as per the manufacturer's protocol. RNA was eluted in 20 μL of DNAse/RNAse-free water and stored at −80° C. until further use. Droplet digital PCR (ddPCR; Bio-Rad Inc., Hercules, Calif., USA) was used to quantify absolute levels of nine miRNA (miR-20a, miR-505, miR-3623p, miR-30d, miR-92a, miR-486, miR-195, miR-93p, miR-151-5p). Prior to ddPCR analysis, RNA was checked for quality using a bioanalyzer assay with a small RNA assay. After quality confirmation, 10 ng of RNA was reverse transcribed using specific miRNA TaqMan assays as per the manufacturer's protocol (Thermo Fisher Scientific Inc., Waltham, Mass., USA). Protocol details can be found in Papa et al 2015. The final PCR product was analyzed using a droplet reader (Bio-Rad Inc., Hercules, Calif., USA). Total positive and negative droplets were quantified, and from this, the concentration of miRNA/μL of the PCR reaction was reported. All reactions were performed in duplicate.

Metabolomic Analysis: The remaining serum was sent to Metabolon (Morrisville, N.C., USA) for metabolomic quantification. Upon arrival, samples were assigned a unique identifier via an automated laboratory system and stored at −80° C. Samples were prepared for subsequent analyses using an automated MicroLab STAR® system (Hamilton Company, Reno, Nev., USA). Proteins were precipitated out of each sample using methanol and a shaker (Glen Mills GenoGrinder 2000), and then centrifuged. The resulting extract was then divided into five fractions for various analyses: 1) two fractions for analysis by two separate reverse phase (RP)/UPLC-MS/MS methods with positive ion mode electrospray ionization (ESI), 2) one for analysis by RP/UPLC-MS/MS with negative ion mode ESI, 3) one for analysis by HILIC/UPLC-MS/MS with negative ion mode ESI, and 4) one reserved for backup. To remove organic solvent, samples were briefly placed on a TurboVap® (Zymark); samples were then stored under nitrogen overnight prior to analyses.

Serum metabolites were quantified using Ultrahigh Performance Liquid Chromatography-Tandem Mass Spectroscopy (UPLC-MS/MS). All methods utilized a Waters ACQUITY UPLC and a Thermo Scientific Q-Exactive high resolution/accurate mass spectrometer interfaced with a heated electrospray ionization (HESI-II) source and Orbitrap mass analyzer operated at 35,000 mass resolution. The sample extract was dried and reconstituted in solvents compatible to each of the listed analyses. Each reconstitution solvent contained a series of standards at fixed concentrations to ensure injection and chromatographic consistency. One aliquot was analyzed using acidic positive ion conditions, chromatographically optimized for more hydrophilic compounds. In this method, the extract was gradient eluted from a C18 column (Waters UPLC BEH C18-2.1×100 mm, 1.7 μm) using water and methanol, containing 0.05% perfluoropentanoic acid (PFPA) and 0.1% formic acid (FA). Another aliquot was also analyzed using acidic positive ion conditions, however it was chromatographically optimized for more hydrophobic compounds. In this method, the extract was gradient eluted from the same afore mentioned C18 column using methanol, acetonitrile, water, 0.05% PFPA and 0.01% FA and was operated at an overall higher organic content. Another aliquot was analyzed using basic negative ion optimized conditions using a separate dedicated C18 column. The basic extracts were gradient eluted from the column using methanol and water, however with 6.5 mM Ammonium Bicarbonate at pH 8. The fourth aliquot was analyzed via negative ionization following elution from a HILIC column (Waters UPLC BEH Amide 2.1×150 mm, 1.7 μm) using a gradient consisting of water and acetonitrile with 10 mM Ammonium Formate, pH 10.8. The MS analysis alternated between MS and data-dependent MS' scans using dynamic exclusion. The scan range varied slighted between methods but covered 70-1000 m/z.

Peak analysis was conducted using a bioinformatics system which consisted of four major components: 1) the Laboratory Information Management System (LIMS—a system used to automate sample accession and preparation, instrumental analysis and reporting, and data analysis), 2) the data extraction and peak-identification software, 3) data processing tools for quality control and compound identification, and 4) a collection of information interpretation and visualization tools.

Raw data was extracted, peak-identified, and QC processed using Metabolon's hardware and software. Compounds were identified by comparison to library entries of purified standards. Biochemical identifications were based on three criteria: 1) retention index (RI) within a narrow RI window of the proposed identification, 2) accurate mass match to the library (+/−10 ppm), and 3) the MS/MS forward and reverse scores between the experimental data and authentic standards. The MS/MS scores were based on a comparison of the ions present in the experimental spectrum to the ions present in the library spectrum. While there may have be similarities between these molecules based on one of these factors, the use of all three data points was utilized to distinguish and differentiate more than 3,300 registered biochemicals.

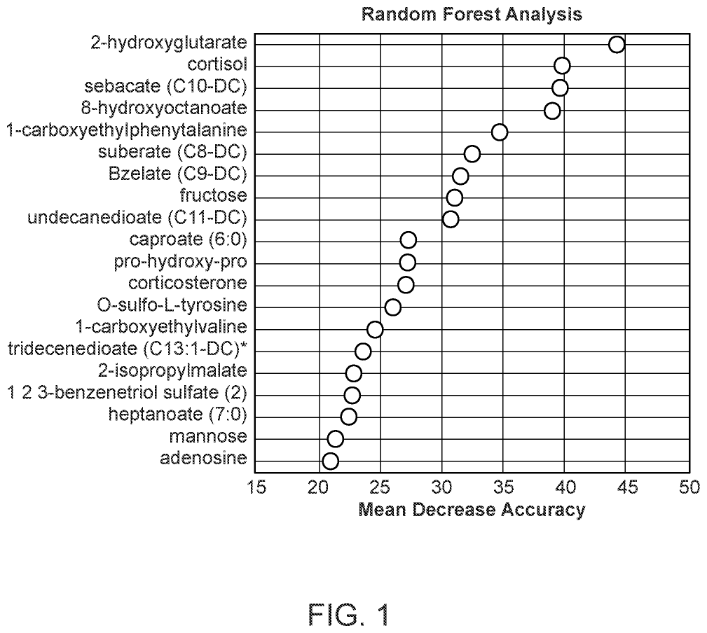

Peaks were quantified using area-under-the-curve. A data normalization step was performed to correct variation resulting from instrument inter-day tuning differences (i.e. variation between pre and postseason analyses). Specifically, each compound was corrected in run-day blocks by registering the medians to equal one (1.00) and normalizing each data point proportionately. Data were then log-transformed. Of the 3,300+ potential biochemicals, 968 metabolites were analyzed at Pre and Post sessions. Of these, 161 showed significant, FDR-corrected, increases or decreases between the Pre and Post sessions (q-value<0.05). Of those 161 metabolites, 40 were selected based on the following criteria: 1) they appeared in the random forest plot (20/40) (FIG. 1 and Table 2) they were hypothesized to change following repetitive HAEs. Based on these criteria, six macromolecule categories were defined: lipids (19/40), energy-related metabolites (5/40), xenobiotics (10/40), amino acids (3/40), carbohydrates (2/40), and nucleotides (1/40) (Table 1). These 40 metabolites were used in all subsequent analyses.

TABLE 1
Metabolite changes across season Metabolite levels between pre- and post-season were assessed using the
Wilcoxon signed-rank test, a nonparametric t-test for paired samples. Metabolites were grouped by super pathway and
FDR corrected by the number of variables in each group (e.g. 19 lipid metabolites). Metabolites with significant q-
values were reported. Negative z-scores indicate an increase from pre- to post-season and positive z-scores indicate a
decrease from pre- to post-season.

| Metabolite | Super Pathway | Sub Pathway | p-value | q-value | z-score |
|---|---|---|---|---|---|
| corticosterone* | lipid | corticosteroid | 0.0002 | 0.0028 | −3.74 |
| cortisol* | lipid | corticosteroid | 0.0001 | 0.0016 | −3.98 |
| cortisone | lipid | corticosteroid | 0.0447 | 0.0447 | −2.01 |
| cortoloneglucuronide (cg) | lipid | corticosteroid | 0.0040 | 0.0194 | 2.88 |
| stearidonate | lipid | long chain polyunsaturated fatty acid | 0.0035 | 0.0194 | −2.92 |
| linoleate3n6 | lipid | long chain polyunsaturated fatty acid | 0.0013 | 0.0091 | −3.22 |
| sebacate* | lipid | fatty acid, dicarboxylate | 0.0001 | 0.0016 | −3.95 |
| linoleate2n6 | lipid | long chain polyunsaturated fatty acid | 0.0097 | 0.0194 | −2.59 |
| dodecadienoate | lipid | fatty acid, dicarboxylate | 0.0068 | 0.0194 | −2.71 |
| azelate* | lipid | fatty acid, dicarboxylate | 0.0003 | 0.0036 | −3.62 |
| suberate* | lipid | fatty acid, dicarboxylate | 0.0002 | 0.0028 | −3.77 |
| 7-hydroxyoctanate | lipid | fatty acid, monohydroxy | 0.0074 | 0.0194 | −2.68 |
| 8-hydroxyoctanate* | lipid | fatty acid, monohydroxy | 0.0001 | 0.0016 | −3.86 |
| undecanedioate* | lipid | fatty acid, dicarboxylate | 0.0004 | 0.0040 | −3.53 |
| caproate* | lipid | medium chain fatty acid | 0.0004 | 0.0040 | 3.53 |
| heptanoate (7:0)* | lipid | medium chain fatty acid | 0.0008 | 0.0072 | 3.35 |
| tridecenedioate* | lipid | fatty acid, dicarboxylate | 0.0003 | 0.0036 | 3.65 |
| 2-hydroxyglutarate* | lipid | fatty acid dicarboxylate | 0.0000 | 0.0000 | −4.14 |
| N-palmitoylserine | lipid | endocannabinoid | 0.0009 | 0.0072 | 3.34 |
| alpha-ketoglutarate | energy | TCA cycle | 0.0386 | 0.0386 | 2.07 |
| citrate | energy | TCA cycle | 0.0192 | 0.0384 | 2.34 |
| aconitate | energy | TCA cycle | 0.0068 | 0.0272 | 2.71 |
| fumarate | energy | TCA cycle | 0.0177 | 0.0384 | 2.37 |
| phosphate | energy | oxidative phosphorylation | 0.0051 | 0.0255 | −2.80 |
| paraxanthine | xenobiotic | xanthine | 0.0220 | 0.0440 | −2.29 |
| 1-methylurate | xenobiotic | xanthine | 0.0034 | 0.0272 | −2.93 |
| 1,3-dimethylurate | xenobiotic | xanthine | 0.0081 | 0.0440 | −2.65 |
| 1,7-dimethylurate | xenobiotic | xanthine | 0.0120 | 0.0440 | −2.51 |
| 1-methylxanthine | xenobiotic | xanthine | 0.0009 | 0.0081 | −3.32 |
| 5-acetylamino-6-amino-3-methyluracil (aam) | xenobiotic | xanthine | 0.0184 | 0.0440 | −2.36 |
| 5-acetylamino-6-formylamino-3-methyluracil (afm) | xenobiotic | xanthine | 0.0123 | 0.0440 | −2.50 |
| O-sulfo-L-tyrosine* | xenobiotic | chemical | 0.0001 | 0.0011 | −3.98 |
| 2-isopropylmalate* | xenobiotic | food component/plant | 0.0089 | 0.0440 | 2.62 |
| 1,2,3-benzenetriol sulfate (2)* | xenobiotic | chemical | 0.0005 | 0.0050 | −3.50 |
| 1 -carboxyethylphenylalanine* | amino acid | phenylalanine metabolism | 0.0002 | 0.0002 | 3.74 |
| prolyl-hydroxy-proline* | amino acid | urea cycle; arginine and proline metabolism | 0.0000 | 0.0000 | −4.17 |
| 1-carboxyethylvaline* | amino acid | leucine, isoleucine and valine metabolism | 0.0002 | 0.0002 | 3.74 |
| fructose* | carbohydrate | fructose, mannose and galactose metabolism | 0.0001 | 0.0001 | 3.86 |
| mannose* | carbohydrate | fructose, mannose and galactose metabolism | 0.0001 | 0.0001 | −3.95 |
| adenosine* | nucleotide | polyamine metabolism | 0.0333 | 0.0333 | 2.13 |

*indicates metabolites that also appeared in the random forest plot.

Virtual Reality (VR) testing: Athletes completed a previously validated Virtual Reality (VR) neurocognitive testing with a 3D TV system (HeadRehab.com) and a head mounted accelerometer for Pre and Post sessions [51-54]. The test included three modules: Spatial Memory, whole body Reaction Time and Balance. These tasks were based on findings from Dr. Alexander Luria for spatial navigation problems following head injuries in World War II Veterans [55-57]. For the Spatial Memory module, participants were shown a randomized virtual pathway including multiple turns to a door along with the return trip. Athletes were instructed to repeat the pathway from their memory using a joystick. The Spatial Memory score was based on correct responses versus the errors as well as based on time to accomplish the test (total allowed 30 sec). Subjects were given 3 trials to complete the task. Failure to complete the task may indicate severe working memory problem with encoding information. Ability to complete the task on third trial may indicate less severe brain trauma and problem with retrieval information.

For Reaction Time module, the participants stood feet shoulder width apart with hands on their hips. They were instructed to move their body, predominantly at ankle joints in the same direction as the virtual room's movements in anterior-posterior (AP) and medio-lateral (ML) directions, and the accelerometer measured response time latency. Time of visually induced postural perturbation and direction of VR room perturbation were randomized to control for learning effect.

For the Balance module, athletes were instructed to hold a modified tandem Romberg position for all trials. The first trial was baseline measure where the virtual room was still. Athletes were scored for the subsequent six trials where the virtual room moved in various (AP and ML) directions, and the deviance of individual's alignment with the virtual room was quantified via an accelerometer followed by computation of area of Center of Pressure. To facilitate interpretation, all scores were scaled such that higher score represents better performance.

In addition to the individual scores from each module, an overall Comprehensive score was calculated by combining the three module scores into a ten-point scale (0 worst and 10 best) [53].

resting-state fMRI: Twenty (20) athletes participated in two Mill sessions consisting of a 10-min eyes-closed rs-fMRI scan with echo-planar imaging and the following parameters: time of echo (TE)=35.8 ms, time of repetition (TR)=2000 ms, flip angle=90°, 72 contiguous 2-mm axial slices in an interleaved order, voxel resolution=2 mm×2 mm×2 mm, matrix size=104×104 and 300 total volumes. The high-resolution T₁ scan using 3D magnetization prepared rapid acquisition gradient recalled echo (3D MPRAGE) sequence was acquired for registration and tissue segmentation purposes with the following parameters: TE=1.77 ms, time of inversion (TI)=850 ms, TR=1700 ms, flip angle=9°, matrix size=320×260×176, voxel size=1 mm×1 mm×1 mm, receiver bandwidth=300 Hz/pixel, and parallel acceleration factor=2.

rs-fMRI data were processed using functions from AFNI [58] and FSL [59, 60] using in-house MATLAB code explained in detail in [61] rs-fMRI BOLD timeseries were processed in the subject's native space and the first four volumes were discarded to remove spin history effects. Structural T₁ images were denoised and segmented into gray matter (GM), white matter (WM) and cerebrospinal fluid (CSF) tissue masks. The 4D BOLD timeseries was then passed through outlier detection, despiking, slice timing correction, volume registration, aligned to the T₁ structural scan, voxel-wise spatial smoothing within tissue masks, scaled to a maximum (absolute value) of 200, and the data were censored to remove outlier timepoints (with the censoring criteria as in [61]. The times series were then detrending using no global signal regression with the following common regressors: (1) very low frequency fluctuations as derived from a bandpass [0.002-0.01 Hz] filter, the six motion parameters and their derivatives, and the voxel-wise local neighborhood (40 mm) mean WM timeseries.

For connectivity analysis on a regional basis, the grey matter brain atlas from [62] was warped to each subject's native space by linear and non-linear registration. This brain parcellation consists of 278 regions of interest (ROIs). Note that data from the cerebellum (including a total of 30 ROIs) were discarded, because the acquired data did not completely cover this structure for all subjects. This resulted in a final GM partition of 248 ROIs. A functional connectivity matrix (namely the functional connectome; FC) was computed for each rs-fMRI scan through correlation of the mean time series from each of the 248 ROIs. The resulting square, symmetric FC matrices were not thresholded or binarized. Each FC matrix was ordered into seven cortical sub-networks, as proposed by Yeo et al. [63], and an additional eighth sub-network comprising sub-cortical regions was added [64]. These networks were: Visual (VIS), Somato-Motor (SM), Dorsal Attention (DA), Ventral Attention (VA), Limbic System (L), Fronto-Parietal (FP), Default Mode Network (DMN) and subcortical regions (SUBC). The fingerprint for FC and these eight networks was calculated by correlating the submatrix corresponding to each of the networks from Pre and Post sessions [61, 65, 66]. The network fingerprint represents the similarity between repeat visits of the same participant.

Statistical Analysis: In order to assess changes across the season, “A” values were calculated for all the aforementioned measures by subtracting Pre-session measures from Post. For rs-fMRI network fingerprint, which represents the similarity between repeat visits of the same participant, was used instead of A values. After quality checks and removing missing data seventeen (17) subjects had both Pre and Post data for all five measurements. All statistical analyses used the software package R [67]. Analyses involved identification of two-way associations, discovery of their overlap as three-way associations, and mediation/moderation testing within a permutation-based framework.

Random Forest analysis: Random forest is an unsupervised classification technique based on an ensemble of decision trees; here, it was used to assess the importance ranking of biochemicals [68]. For a given decision tree, a random subset of the data, with identifying true class information, was selected to build the tree (“bootstrap sample” or “training set”), and then the remaining data, the “out-of-bag” (OOB) variables, were passed down the tree to obtain a class prediction for each sample. This process was repeated thousands of times to produce the forest. The final classification of each sample was determined by computing the class prediction frequency for the OOB variables over the whole forest. When the full forest was grown, the class predictions were compared to the true classes, generating the “OOB error rate” as a measure of prediction accuracy. Thus, the prediction accuracy was an unbiased estimate of how well a sample class was predicted in a new data set. To determine which biochemicals made the largest contribution to the classification, a variable importance measure, mean Decrease Accuracy (MDA), was computed. MDA was determined by randomly permuting a variable, running the observed values through the trees, and then reassessing the prediction accuracy. If a variable was not important, the procedure produced little change in the accuracy of the class prediction (permuting random noise gave random noise). By contrast, if a variable was important to the classification, the prediction accuracy dropped; this was recorded as the MDA.

Wilcoxon signed-rank test for across-season miRNA, metabolite, and VR score analysis: Data were first tested for normality and equal variances using the Shapiro-Wilks test and Bartlett's test. Because 1) data were not normally distributed (Shapiro-Wilks test; p≥0.05) and/or data did not have equal variances (Bartlett's test; p-value≥0.05) and 2) data were paired by subject across season, a Wilcoxon signed-rank test was utilized to assess across-season changes. Changes were analyzed at a significance level of 0.05. If p-value<0.05, metabolites were then grouped based on super pathway and FDR-corrected using the Benjamini-Hochberg method for multiple comparisons (Table 1). Changes were considered significant if q-value≤0.05.

Linear regression analyses: Linear regressions were conducted to assess significant interactions between VR scores, miRNAs, metabolites, and rs-fMRI networks where VR score (i.e., behavior) was always the dependent variable (Y). When regressing miRNA and metabolite, metabolite was the designated as the dependent variable; this decision was based on previous findings where miRNA levels were elevated in this cohort at preseason when compared to controls [69]. After each initial regression analysis, Cook's distance was calculated to reveal outliers which drastically influenced the regression (i.e., large shift in (3) [70, 71]. Outliers were removed if Cook's distance>4/n and regressions were then re-run [72]. Regressions were considered significant if p-value<0.05 and standardized beta coefficients (Std. β) and adjusted R² values (R_{adj}²) were also reported.

Three-way associations: All two-way associations with p≤0.05 were used to build three-way associations. The measurement matrices Xₜ, Yₜ, Zₜ defined below schematize the variables used, such as the network fingerprint, VR scores, miRNAs and metabolites.

$${X}_{t} = [ \begin{matrix}{x}_{t , 1 , 1} & & {x}_{t , N , 1} \\ ⋮ & … & ⋮ \\ {x}_{t , 1 , S} & & {x}_{t , N , S}\end{matrix} ] , \\ {Y}_{t} = [ \begin{matrix}{y}_{t , 1 , 1} & & {Y}_{t , M , 1} \\ ⋮ & … & ⋮ \\ {y}_{t , 1 , S} & & {y}_{t , M , S}\end{matrix} ] , \\ {Z}_{t} = [ \begin{matrix}{z}_{t , 1 , 1} & & {z}_{t , P , 1} \\ ⋮ & … & ⋮ \\ {z}_{t , 1 , S} & & {z}_{t , P , S}\end{matrix} ]$$

where N is the total number of variables in matrix Xₜ, M and P are the total number of variables for matrices Yₜ and Zₜ respectively. S is the number of participants and the matrices were measured at two time points t=1 represents Pre and t=2 represents Post-season measurements.

Across season measures for VR scores, miRNAs and metabolites were calculated as

ΔX=X₂−X₁

ΔY=Y₂−Y₁

ΔZ=Z₂Z₁

Note that, for rs-fMRI instead of difference measures network fingerprint was computed by correlating the data at two time-points.

In what follows, Δyⱼ˜Δxᵢ will be used to denote the significant two-way association between any two variables (following the procedure described above).

In order to quantify three-way associations, the following steps (A-C, below) were performed

$$Step A \text{:} Δ {y}_{j} \text{∼} Δ {x}_{i} \{ \begin{matrix}∀ i ∈ \{ 1 , … , N \} \\ ∀ j ∈ \{ 1 , … M \}\end{matrix}$$

Two-way associations were performed between all variables Δyⱼ and Δxᵢ from matrices ΔY and ΔX.

$$Step B \text{:} Δ {z}_{k} \text{∼} Δ {y}_{j} \{ \begin{matrix}∀ i ∈ \{ 1 , … M \} \\ ∀ k ∈ \{ 1 , … P \}\end{matrix}$$

Two-way associations were performed between all variables Δzₖ and Δyⱼ from matrices ΔZ and ΔY.

$$Step C \text{:} Δ {z}_{k} \text{∼} Δ {x}_{i} \{ \begin{matrix}∀ i ∈ \{ 1 , … , N \} \\ ∀ k ∈ \{ 1 , … P \}\end{matrix}$$

Two-way associations were performed between all variables Δzₖ and Δxᵢ from matrices ΔZ and ΔX.

Three-way associations between any three variables were formed if all three above steps resulted in significant two-way associations for the common variables as below

Δxᵢ˜Δyⱼ˜Δzₖ˜Δxᵢ

Permutation-based mediation analysis: Based on results from the linear regression analyses, data were prepared for mediation analysis by first assessing three-way associations. To be included in mediation analysis, all paths must have met significance (p-value<0.05) to form a three-way association. Paths were as follows: A) miRNA (X)→metabolite (Y), B) metabolite (Y)→VR score (Z), and C) miRNA (X)→VR score (Z). Cook's outliers were removed across all regressions to achieve the same set of subjects, and regressions were re-run with common subjects. Mediation seeks to clarify the causal relationship between the independent variable (X) and dependent variable (Y) with the inclusion of a third variable mediator (M). The mediation model proposes that instead of a direct causal relationship between X and Y, the X influences M which then influences the Y. Beta coefficients (β) and standard error (se) terms from the following linear regression equations are used to calculate the Sobel p-value and mediation effect percentage (T_{eff}):

Step 1(Path A):M=β₀+β_{1A}(X)+∈_{A}

Step 2(Path B):Y=β₀+β_{1B}(M)+∈_{B}

Step 3(Path C, model 1):Y=β₀+β_{1,1C}(X)+∈_{1C}

Step 4(Path C, model 2):Y=β₀+β_{1,2C}(X)+β_{2,2C}(M)+∈_{2C}

Sobel's test was then used to test if β_{1,2C} was significantly lower than β_{1,1C} using the following equation:

$$Sobel z \text{-} score = \frac{( {β}_{1 , 1 C} - {β}_{1 , 2 C} )}{\sqrt{[ {( {β}_{2 , 2 C} )}^{2} {( {ϵ}_{1 A} )}^{2} ] + [ {( {β}_{1 A} )}^{2} {( {ϵ}_{2 C} )}^{2} ]}} \qquad ( 3 )$$

Using a standard 2-tail z-score table, the Sobel p-value was determined from Sobel z-score. Mediation effect percentage (T_{eff}) was calculated using the following equation:

$${T}_{eff} = 100 * \frac{( {β}_{1 A} * {β}_{2 , 2 C} )}{( {β}_{1 A} * {β}_{2 , 2 C} ) + [ {β}_{1 , 1 C} - ( {β}_{1 A} * {β}_{2 , 2 C} ) ]} \qquad ( 4 )$$

In order to control for the occurrence of false positives due to multiple hypotheses testing, permutation-based mediation analysis was performed for all three-way associations. Hypothesis-directed mediation results were considered significant if p_{Sobel}^{perm}<0.05, T_{eff} was >50%, and T_{eff} for the control mediation was <30%.

Permutation-based moderation analysis: For this study, permutation-based moderation analysis was performed for all three-way associations involving rs-fMRI networks. All three-way associations were also tested for moderations. Moderation model proposes that the strength and direction of the relationship between independent variable (IV) and dependent variable (DV) is controlled by the moderator variable (M). Moderation Analysis was performed by fixing ΔVR scores as the dependent variable (DV). The independent variable (IV) and moderator (M) were chosen from network fingerprints, ΔmiRNAs and Δmetabolites. The moderation is characterized by the interaction term between IV and M in the linear regression equation as given below:

DV=β₀+β₁IV+β₂M+β₃(IV*M)+∈

Moderation was significant if p_{β}_{₃}≤0.05 and p_{F≤}0.05, where p 0.05 indicates that β₃ is significantly different than zero using a t-test and p_{F} is the p-value associated with the overall F-test for the regression equation suggesting that the overall linear relationship is significant.

Head acceleration event monitoring: Head acceleration events (HAEs) were monitored at all contact practice sessions (max=53) using the BodiTrak sensor system from The Head Health Network [51]. Sensors were mounted in each active player's helmet prior to contact practice (no games were monitored). Sensor outputs included peak translational acceleration (PTA; G-units) and impact location. HAEs were quantified as 1) cumulative hits exceeding 25 G and 80 G (cHAE_{25G} and cHAE_{80G}; Eq. 1) and 2) cumulative hits exceeding 25 G and 80 G normalized to the total number of sessions per player (aHAE_{25G} and aHAE_{80G}; Eq. 2). The G-unit thresholds (Th) were selected based on previous reports of impacts related to brain health and injury [73, 74].

$$\begin{matrix}c H A {E}_{T h , i} = {\sum }_{k = 1}^{N} u ( P T {A}_{k , i} - T h ) \\ where u ( x ) = \{ \begin{matrix}1 & if x > 0 \\ 0 & if x \leq 0\end{matrix} & ( 1 ) \\ {aHAE}_{Th , i} = \frac{{cHAE}_{Th , i}}{{sessions}_{i}} & ( 2 )\end{matrix}$$

### Results

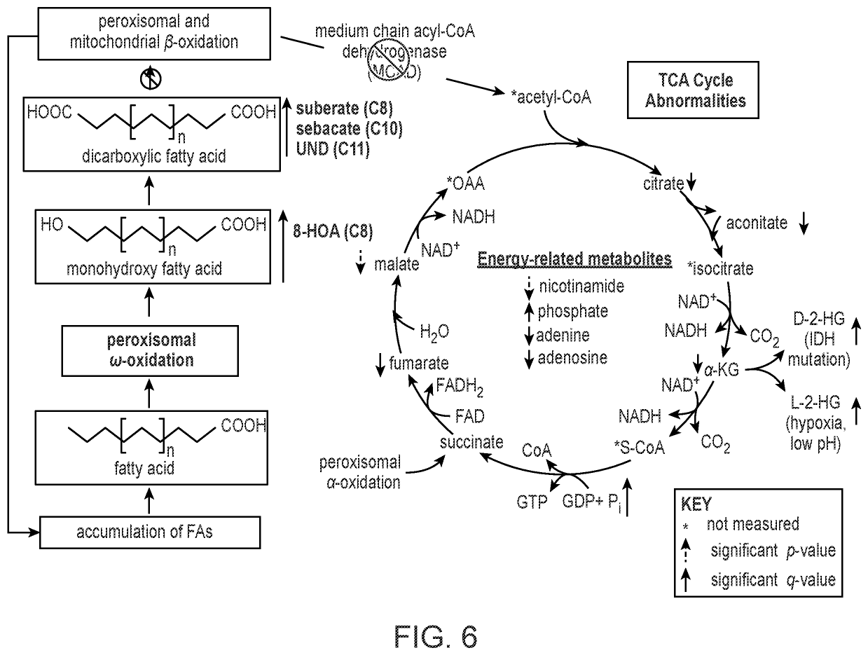

Summary of Results: Applicant identified a set of eight metabolites with significant mediating and moderating effects on behavior: 8-hydroxyoctanoate, tridecenedioate, 2-hydroxyglutarate, undecanedioate, sebacate, suberate, heptanoate, and adenosine (FIG. 6). Additionally, six metabolites in biochemical relation with the aforementioned metabolites were observed to change across the season of football participation: citrate, aconitate, alpha-ketoglutarate, fumarate, phosphate, and adenine. Together, these changes indicate a state of mitochondrial dysfunction. In particular, fatty acid beta-oxidation was impaired, marked by an increase in medium chain monohydroxy and dicarboxylic fatty acids (suberate, sebacate, 8-hydroxyoctanoate, and undecanedioate) and a subsequent decrease in TCA cycle metabolites (citrate, aconitate, fumarate, and alpha-ketoglutarate). Additionally, there was a decrease in the monosaturated long chain dicarboxylic acid, tridecenedioate—which is hypothesized to act as an ROS and free radical scavenger in cases of increased ROS production due to injury. There was also a decrease in adenosine, a nucleoside which is critically important for the formation of ATP. 2-hydroxyglutrate (2-HG), a small alpha dicarboxylic fatty acid, increased. 2-HG is an established oncometabolite due to its accumulation following IDH1/2 mutations; however, it has been observed to increase during states of hypoxia and low pH. Lastly, heptanoate, a monohydroxy carboxylic fatty acid, decreased—opposite of what was observed for the other monohydroxy and dicarboxylic fatty acids. Interestingly, increased levels of heptanoate, via metabolism of triheptanoin, has been marked as beneficial in the treatment of beta-oxidation related metabolic disorders. Therefore, a decrease in heptanoate could be detrimental.

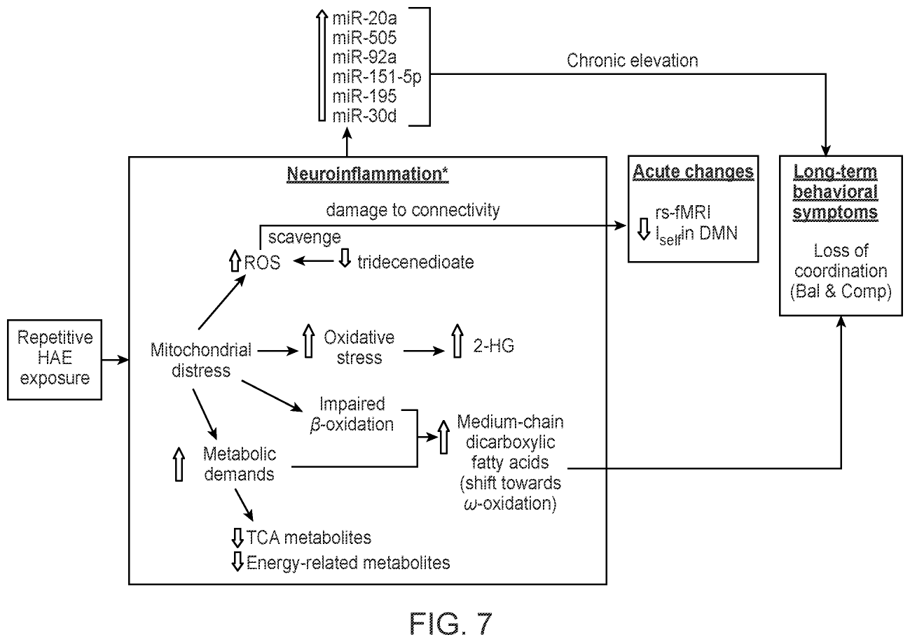

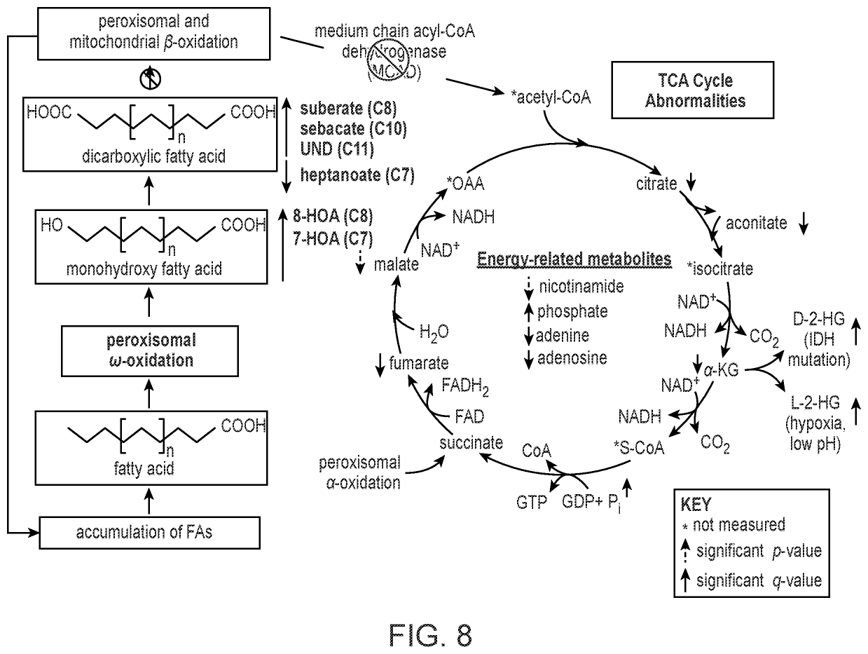

Taken together, this subset of metabolites is indicative of mitochondrial distress resulting from repetitive head acceleration events (FIG. 7). Increased dicarboxylic fatty acids, along with decreased TCA metabolites, points specifically to dysfunctional beta-oxidation and increased levels of 2-HG are indicative of oxidative stress. Additionally, a decrease in a monounsaturated long-chain fatty acid (tridecenedioate) may reflect an increase in ROS which can cause significant damage to structures at the molecular, cellular, and network level—observed as a decreased in Itself in the DMN. Chronic elevation of neuroinflammatory-related miRNAs (miR-20a, miR505, miR-30d, miR-92a, miR-151-5p, miR-195) together with acute brain network alterations and repetitive fluctuations of metabolites may impact long-term changes in cognition, behavior, and mental well-being. At the collegiate level, changes in metabolomic and miRNA signals were significantly associated with computational behavior performance, even in the absence standalone behavioral changes. Therefore, behavior alone is not a satisfactory biomarker for neurophysiological damage.

The presented findings demonstrate a novel metabolomic biomarker for subconcussive mitochondrial beta-oxidation dysfunction using computational behavior, elevated miRNA, and advanced neuroimaging for initial validation. This biomarker has vast applications for concussion diagnostics field and can also be used to test the efficacy of novel brain injury therapeutics.

### Results Integrating Multiscale Data

Random forest plot revealed biochemically important metabolites: Of the 1300+ metabolites analyzed, 20 metabolites with the highest predictive power to distinguish between pre- and postseason (i.e. importance) were plotted (FIG. 1). Of these metabolites 11/20 (55%) were lipids, 3/20 (15%) were amino acids, 2/20 (10%) were carbohydrates, 3/20 (15%) were xenobiotics, and 1/20 (5%) was an amino acid. The metabolite with the highest predictive power was 2-HG, followed by three other lipids. All 20 metabolites from the random forest plot were included in additional analyses.

Wilcoxon signed-rank analyses revealed significant across-season metabolite changes: Wilcoxon signed-rank tests were conducted to determine which miRNA, metabolites, and VR scores significantly fluctuated between pre and postseason. There were no significant (p-value>0.05) changes in miRNA levels and VR scores between pre and postseason. Of the 968 metabolites analyzed, 259 significantly increased or decreased (p-value≤0.05) between pre and postseason. Following FDR correction, 161 significantly increased or decreased (q-value≤0.05). Of those 161 metabolites, 40 were selected based on the criteria described in Metabolomic analysis (FIG. 1). Of the 40 metabolites, 26 significantly increased (negative z-score) and 14 decreased (negative z-score) (Table 1). 48% of the metabolites were lipids and 52% fell in another category (12.5% energy-related, 25% xanthine, 7.5% amino acid, 5% carbohydrate, and 2.5% nucleic acid). Of the lipids, 74% were FAs and 26% were other lipid types. Overall, 35% of the metabolites were FAs and 65% fell in another category. Of the FAs, 79% significantly increased from pre- to postseason. Additionally, all TCA-related metabolites (α-ketoglutarate, citrate, aconitate, and fumarate) decreased from pre- to postseason and all of the xanthine metabolites increased.

Summary of preseason and across-season interactions between miRNA, metabolites, VR scores, and rs-fMRI network: Linear regressions were conducted to assess interactions between four VR scores (Bal, RT, SM, and Comp), nine miRNAs (miR-20a, miR-505, miR-3623p, miR-30d, miR-92a, miR-486, miR-92a, miR-93p, and miR-151-5p), five HAE metrics (sessions, cHAE_{25G}, aHAE_{25G}, cHAE_{80G}, and aHAE_{80G}), and 40 metabolites (Table 1). At preseason, there were nine significant interactions between VR scores and miRNAs, 15 between VR scores and metabolites, and 36 between miRNAs and metabolites (p≤0.05 following Cook's outlier removal) (Tables 2A-2C). Across-season (postseason-preseason), there were 12 significant interactions between VR scores and miRNAs, 20 between VR scores and metabolites, and 31 between miRNAs and metabolites (p≤0.05 following Cook's outlier removal) (Table 3). In general, both pre and across-season analyses revealed negative relationships between VR scores and miRNAs and positive relationships between VR scores and metabolites (exceptions: cortoloneglucuronide, corticosterone, adenosine, and tridecenedioate). Relationships between miRNAs and metabolites varied with the majority being negative (79%).

TABLE 2
Prcscason linear regression results. Cook's outliers were removed prior to regression analysis and
the number of outliers removed in each regression is listed as the ratio of outliers to the total n.
Adjusted R (Radi.) *s reported for each regression and relays the amount of variance in Y that can
be explained by X. />-Values are reported at a significance level of 0.05. (A) Significant interactions
between VR scores and miRNA levels. (B) Significant interactions between VR scores and
metabolite levels. (C) Significant interactions between miRNA and metabolite levels.
A

| VR task (Y) | miRNA (X) | Std. β | R_{adj}² | p-value | Cook's outliers |
|---|---|---|---|---|---|
| Comprehensive | 20a | −0.631 | 0.251 | 0.014 | 0/20 |
| Comprehensive | 92a | −0.444 | 0.157 | 0.039 | 0/22 |
| Comprehensive | 505 | −0.471 | 0.181 | 0.031 | 0/21 |
| Comprehensive | 30d | −0.605 | 0.333 | 0.004 | 1/22 |
| Comprehensive | 151-5p | −0.661 | 0.390 | 0.001 | 1/22 |
| Balance | 505 | −0.721 | 0.476 | 0.000 | 1/21 |
| Balance | 151-5p | −0.579 | 0.294 | 0.012 | 4/22 |
| Reaction Time | 195 | −0.472 | 0.182 | 0.031 | 1/22 |
| Spatial Memory | 486 | −0.546 | 0.215 | 0.013 | 2/22 |

B

| VR task (Y) | Metabolite (X) | Std. β | R_{adj}² | p-value | Cook's outliers |
|---|---|---|---|---|---|
| Comprehensive | 2-hydroxyglutarate | 0.760 | 0.558 | 0.000 | 0/23 |
| Comprehensive | cortisol | 0.484 | 0.191 | 0.031 | 3/23 |
| Comprehensive | sebacate | 0.523 | 0.237 | 0.013 | 1/23 |
| Comprehensive | 8-hydroxyoctanate | 0.612 | 0.343 | 0.002 | 1/23 |
| Comprehensive | undecanedioate | 0.528 | 0.242 | 0.012 | 1/23 |
| Balance | 2-hydroxyglutarate | 0.475 | 0.183 | 0.034 | 3/23 |
| Balance | cortoloneglucuronide | −0.536 | 0.249 | 0.012 | 2/23 |
| Balance | sebacate | 0.498 | 0.208 | 0.022 | 2/23 |
| Balance | suberate | 0.425 | 0.140 | 0.049 | 1/23 |
| Balance | 8-hydroxyoctanate | 0.560 | 0.278 | 0.008 | 2/23 |
| Balance | undecanedioate | 0.652 | 0.393 | 0.002 | 3/23 |
| Reaction Time | 2-hydroxyglutarate | 0.525 | 0.241 | 0.010 | 0/23 |
| Reaction Time | sebacate | 0.516 | 0.229 | 0.014 | 1/23 |
| Reaction Time | 8-hydroxyoctanate | 0.503 | 0.218 | 0.014 | 0/23 |
| Reaction Time | undecanedioate | 0.547 | 0.262 | 0.010 | 2/23 |

C

| Metabolite (Y) | miRNA (X) | Std. β | R_{adj}² | p-value | Cook's outliers |
|---|---|---|---|---|---|
| 2-hydroxglutarate | 20a | −0.529 | 0.235 | 0.024 | 2/20 |
| sebacate | 20a | −0.486 | 0.191 | 0.035 | 1/20 |
| 8-hydroxyoctanate | 20a | −0.619 | 0.347 | 0.005 | 1/20 |
| undecanedioate | 20a | −0.589 | 0.306 | 0.010 | 2/20 |
| heptanoate (7:0) | 20a | −0.470 | 0.175 | 0.042 | 1/20 |
| citrate | 505 | −0.450 | 0.156 | 0.050 | 2/21 |
| 2-hydroxglutarate | 505 | −0.657 | 0.400 | 0.002 | 1/21 |
| 8-hydroxyoctanate | 505 | −0.508 | 0.219 | 0.019 | 1/21 |
| undecanedioate | 505 | −0.478 | 0.183 | 0.038 | 2/21 |
| 1-carboxyethylphenylalanine | 505 | −0.514 | 0.218 | 0.029 | 3/21 |
| heptanoate (7:0) | 505 | −0.458 | 0.168 | 0.037 | 0/21 |
| cortisone | 3623p | −0.567 | 0.286 | 0.007 | 1/22 |
| phosphate | 3623p | 0.572 | 0.287 | 0.011 | 3/22 |
| paraxanthine | 3623p | 0.716 | 0.485 | 0.000 | 2/22 |
| 1,7-dimethylurate | 3623p | 0.459 | 0.167 | 0.042 | 2/22 |
| 1-methybranthine | 3623p | 0.513 | 0.222 | 0.021 | 2/22 |
| 1 2 3-benzenetriol sulfate | 3623p | 0.721 | 0.495 | 0.000 | 1/22 |
| cortisone | 30d | −0.509 | 0.223 | 0.015 | 0/22 |
| cortoloneglucuronide | 30d | 0.470 | 0.229 | 0.032 | 1/22 |
| 2-hydroxglutarate | 30d | −0.450 | 0.156 | 0.053 | 3/22 |
| heptanoate (7:0) | 30d | −0.445 | 0.158 | 0.038 | 0/22 |
| citrate | 92a | −0.473 | 0.185 | 0.026 | 0/22 |
| 8 hydroxyoctanate | 92a | −0.552 | 0.268 | 0.010 | 1/22 |
| heptanoate (7:0) | 92a | −0.567 | 0.284 | 0.009 | 2/22 |
| cortoloneglucuronide | 486 | −0.544 | 0.257 | 0.013 | 2/22 |
| citrate | 195 | −0.486 | 0.198 | 0.022 | 0/22 |
| afm | 195 | 0.248 | 0.210 | 0.028 | 3/22 |
| 8-hydroxyoctanate | 195 | −0.443 | 0.156 | 0.039 | 0/22 |
| heptanoate (7:0) | 195 | −0.538 | 0.252 | 0.012 | 1/22 |
| 1 2 3-benzenetriol sulfate | 195 | 0.740 | 0.522 | 0.000 | 2/22 |
| aconitate | 151-5p | −0.429 | 0.137 | 0.051 | 0/22 |
| 2-hydroxglutarate | 151-5p | −0.516 | 0.226 | 0.02 | 2/22 |
| azelate | 151-5p | −0.445 | 0.156 | 0.043 | 1/22 |
| 8-hydroxyoctanate | 151-5p | −0.554 | 0.270 | 0.009 | 1/22 |
| undecanedioate | 151-5p | −0.575 | 0.293 | 0.008 | 2/22 |
| 1-carboxyethylphenylalanine | 151-5p | −0.490 | 0.198 | 0.028 | 2/22 |

TABLE 3
Across-season linear regression results. Variables are expressed as the difference
between postseason and preseason values (post-pre = Δ). Cook's outliers were
removed prior to regression analysis and the number of outliers removed in each
regression is listed as the ratio of outliers to the total n. Adjusted R² (R_{adj.}²) is
reported for each regression and relays the amount of variance in Y that can be
explained by X. p-Values are reported at a significance level of 0.05. (A) Significant
interactions between ΔVR scores and ΔmiRNA levels (IV). (B) Significant
interactions between ΔVR scores and Δmetabolite levels. (C) Significant
interactions between ΔmiRNA and Δmetabolite levels.
A

| VR task (Y) | miRNA (X) | Std. β | R_{adj}² | p-value | Cook's outliers |
|---|---|---|---|---|---|
| Comprehensive | 505 | −0.649 | 0.387 | 0.003 | 1/20 |
| Comprehensive | 30d | −0.586 | 0.306 | 0.007 | 0/20 |
| Comprehensive | 92a | −0.479 | 0.186 | 0.033 | 0/20 |
| Comprehensive | 195 | −0.436 | 0.145 | 0.050 | 0/20 |
| Comprehensive | 151-5p | −0.655 | 0.395 | 0.002 | 1/20 |
| Balance | 505 | −0.651 | 0.389 | 0.003 | 1/20 |
| Balance | 30d | −0.507 | 0.213 | 0.027 | 1/20 |
| Reaction Time | 20a | −0.482 | 0.185 | 0.043 | 0/18 |
| Reaction Time | 505 | −0.544 | 0.254 | 0.016 | 1/20 |
| Reaction Time | 30d | −0.483 | 0.190 | 0.031 | 0/20 |
| Reaction Time | 92a | −0.537 | 0.249 | 0.015 | 0/20 |
| Reaction Time | 151-5p | −0.651 | 0.323 | 0.007 | 1/20 |

B

| VR task (Y) | miRNA (X) | Std. β | R_{adj}² | p-value | Cook's outliers |
|---|---|---|---|---|---|
| Comprehensive | 505 | −0.649 | 0.387 | 0.003 | 1/20 |
| Comprehensive | 30d | −0.586 | 0.306 | 0.007 | 0/20 |
| Comprehensive | 92a | −0.479 | 0.186 | 0.033 | 0/20 |
| Comprehensive | 195 | −0.436 | 0.145 | 0.050 | 0/20 |
| Comprehensive | 151-5p | −0.655 | 0.395 | 0.002 | 1/20 |
| Balance | 505 | −0.651 | 0.389 | 0.003 | 1/20 |
| Balance | 30d | −0.507 | 0.213 | 0.027 | 1/20 |
| Reaction Time | 20a | −0.482 | 0.185 | 0.043 | 0/18 |
| Reaction Time | 505 | −0.544 | 0.254 | 0.016 | 1/20 |
| Reaction Time | 30d | −0.483 | 0.190 | 0.031 | 0/20 |
| Reaction Time | 92a | −0.537 | 0.249 | 0.015 | 0/20 |
| Reaction Time | 151-5p | −0.651 | 0.323 | 0.007 | 1/20 |

C

| Metabolite (Y) | miRNA (X) | Std. β | R_{adj}² | p-value | Cook's outliers |
|---|---|---|---|---|---|
| azelate | 20a | −0.636 | 0.365 | 0.006 | 1/18 |
| sebacate | 505 | −0.591 | 0.309 | 0.010 | 2/20 |
| azelate | 505 | −0.662 | 0.404 | 0.003 | 20/20 |
| suberate | 505 | −0.600 | 0.320 | 0.008 | 2/20 |
| 8-hydroxyoctanate | 505 | −0.485 | 0.188 | 0.041 | 2/20 |
| undecanedioate | 505 | −0.604 | 0.326 | 0.008 | 2/20 |
| heptanoate (7:0) | 505 | −0.514 | 0.218 | 0.029 | 2/20 |
| tridecenedioate | 505 | 0.450 | 0.156 | 0.050 | 1/20 |
| phosphate | 3623p | −0.495 | 0.163 | 0.050 | 2/20 |
| linoleate3n6 | 3623p | 0.509 | 0.210 | 0.037 | 3/20 |
| heptanoate (7:0) | 3623p | −0.543 | 0.256 | 0.013 | 0/20 |
| sebacate | 30d | −0.528 | 0.239 | 0.017 | 0/20 |
| heptanoate (7:0) | 30d | −0.618 | 0.346 | 0.005 | 1/20 |
| adenosine | 30d | 0.550 | 0.259 | 0.018 | 2/20 |
| sebacate | 92a | −0.734 | 0.510 | 0.001 | 2/20 |
| azelate | 92a | −0.689 | 0.437 | 0.003 | 4/20 |
| suberate | 92a | −0.622 | 0.346 | 0.008 | 3/20 |
| undecanedioate | 92a | −0.641 | 0.375 | 0.004 | 2/20 |
| heptanoate (7:0) | 92a | −0.523 | 0.234 | 0.018 | 0/20 |
| O-sulfo-L-tyrosine | 92a | −0.506 | 0.215 | 0.023 | 0/20 |
| adenosine | 92a | 0.496 | 0.199 | 0.036 | 2/20 |
| sebacate | 195 | −0.545 | 0.256 | 0.016 | 1/20 |
| suberate | 195 | −0.476 | 0.175 | 0.053 | 3/20 |
| 8-hydroxyoctanate | 195 | −0.632 | 0.364 | 0.004 | 1/20 |
| undecanedioate | 195 | −0.530 | 0.239 | 0.020 | 1/20 |
| heptanoate (7:0) | 195 | −0.667 | 0.412 | 0.002 | 1/20 |
| O-sulfo-L-tyrosine | 195 | −0.530 | 0.238 | 0.020 | 1/20 |
| 7-hydroxyoctanate | 93p | 0.535 | 0.247 | 0.018 | 1/20 |
| stearidonate | 151-5p | 0.556 | 0.263 | 0.020 | 3/20 |
| 8-hydroxyoctanate | 151-5p | −0.480 | 0.185 | 0.038 | 1/20 |
| heptanoate (7:0) | 151-5p | −0.562 | 0.273 | 0.015 | 2/20 |

Linear regression following Cook's outlier removal was also used to assess significant (p≤0.05) two-way associations between functional connectome (FC) and eight network fingerprints (Vis, SM, DA, VA, L, FP, DMN and SUBC), four ΔVR scores (Balance, Reaction Time, Spatial Memory and Comprehensive), nine ΔmiRNA (miR-20a, miR-505, miR-3623p, miR-30d, miR-92a, miR-486, miR-92a, miR-93p, and miR-151-5p), 40 Δmetabolites. Secondary analyses included assessments of these four categories of measure against the five HAE metrics (sessions, cHAE_{25G}, aHAE_{25G}, cHAE_{80G}, and aHAE_{80G}). The common significant pairwise associations were then used to build three-way associations. Three-way associations were formed from the common significant pairwise associations between any three variables Δxᵢ, Δyⱼ and Δzₖ where these could be any of the network fingerprints, ΔVR scores, ΔmiRNAs and/or Δmetabolites (see Methods for details).

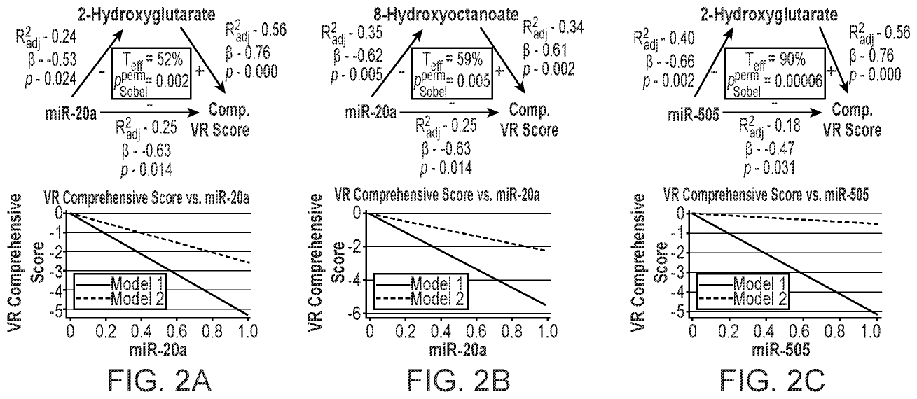

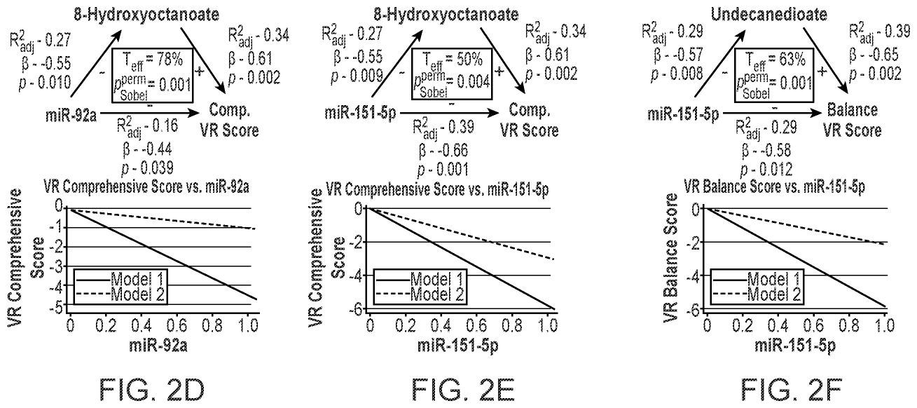

Mediation analyses revealed causal mediations at preseason and across-season: Mediation analyses were performed to assess the causal influence of metabolites on the interaction between miRNA and VR scores. Specifically, the hypothesized mediation was directed so miRNA was the independent variable, metabolite was the mediator, and VR score was the dependent variable. Of the 18 preseason three-way associations, 11 survived following combined removal of Cook's outliers across all paths (A, B, and C)—ensuring the same set of subjects were being analyzed. Mediation analysis revealed six significant preseason mediations (Sobel p-value<0.05, T_{eff}≥50%). FIG. 2 depicts each significant mediation, including the directionality and significance of each regression interaction. The graphs illustrate the slopes of linear models 1 (IV→DV) versus 2 (IV+M→DV). In each case, the slope of model 1 was larger than that of model 2, indicating that the metabolite (mediator) significantly impacted the relationship between the miRNA (independent variable) and VR score (dependent variable). The directionality of the linear regression relationships was common across all preseason mediations-negative between miRNA and metabolite, negative between miRNA and VR score, and positive between metabolite and VR score. No mediations met both a significant p-value and a T_{eff} greater than 50% when metabolite was the IV, miRNA was the M, and VR score was the DV (p-value>0.048, T_{eff}<30%).

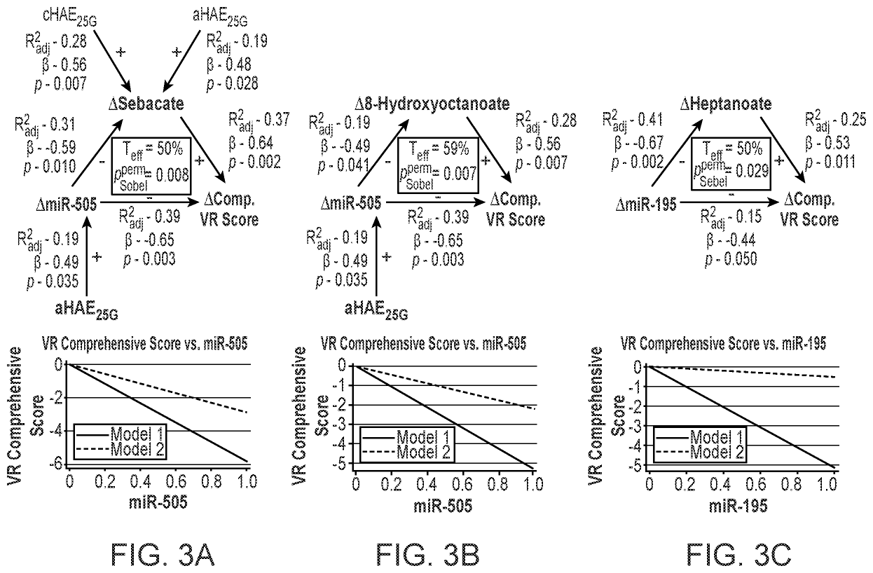

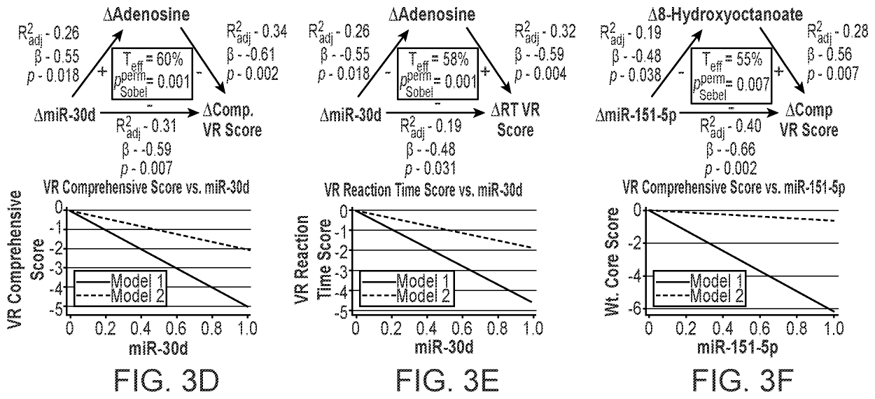

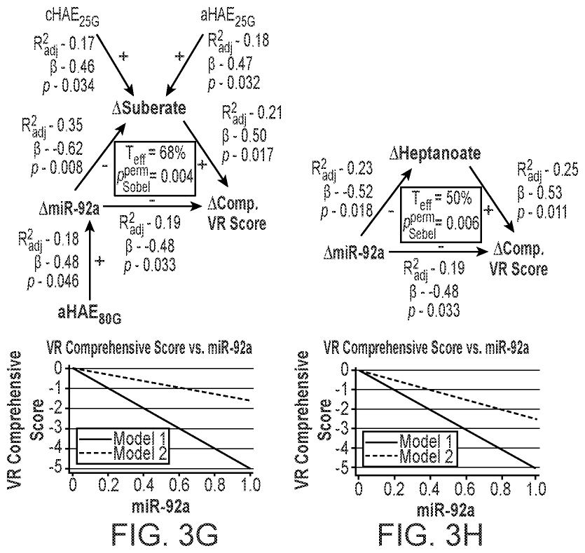

Across-season mediation analysis revealed eight mediations (Sobel p-value<0.05; T_{eff}>50%). FIG. 3A-3H visualizes each across-season mediation, including the directionality of each interaction. The plots depict the slopes of linear models 1 (IV→DV) versus 2 (IV+M→DV). In each case, the slope of model 1 was larger than that of model 2. Mediations depicted in FIGS. 3A-3C and 3F-3I share the same directionality across regressions: negative between miRNA and metabolite, positive between metabolite and VR score, and negative between miRNA and VR score. In 3D-3E, the interactions between miRNA (miR-30d) and metabolite (adenosine), as well as metabolite and VR score (Comp), were opposite of those depicted in 3A-3C. No mediations were significant when metabolite was the IV, miRNA was the M, and VR score was the DV (p-value>0.133, T_{eff}<48%).

In total, there were 14 significant mediations (p-value<0.05) comprised of seven metabolites (2-HG, 8-HOA, UND, sebacate, suberate, heptanote, adenosine), six miRNAs (miR-20a, miR-505, miR-92a, miR-151-5p, miR-195, and miR-30d), and three VR scores (Comp, Bal, and RT). Of the seven metabolites, six were FAs. Five of the six FAs (2-HG, 8-HOA, UND, sebacate, and suberate) significantly increased from pre- to postseason, while heptanoate and adenosine, a nucleoside, significantly decreased (Table 1). Interestingly, Comp, 8-HOA, miR-505, miR-92a, and miR-151-5p were observed in both pre- and across-season mediations.

Moderation results: The three-way associations identified were then tested for moderation analysis across network fingerprint, ΔVR, Δmetabolite and ΔmiRNA. Two moderation results were observed meeting permutation criteria.

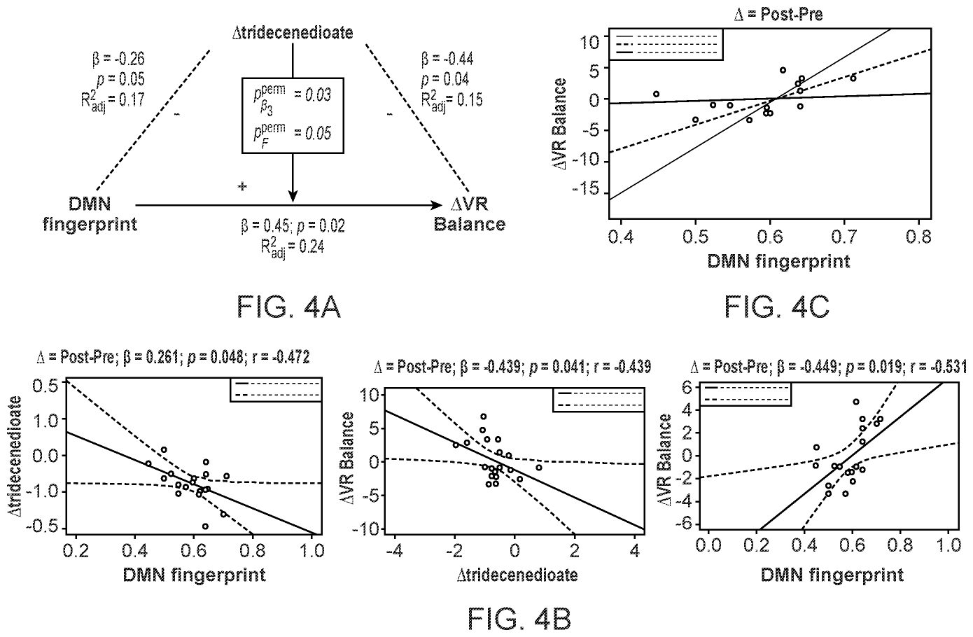

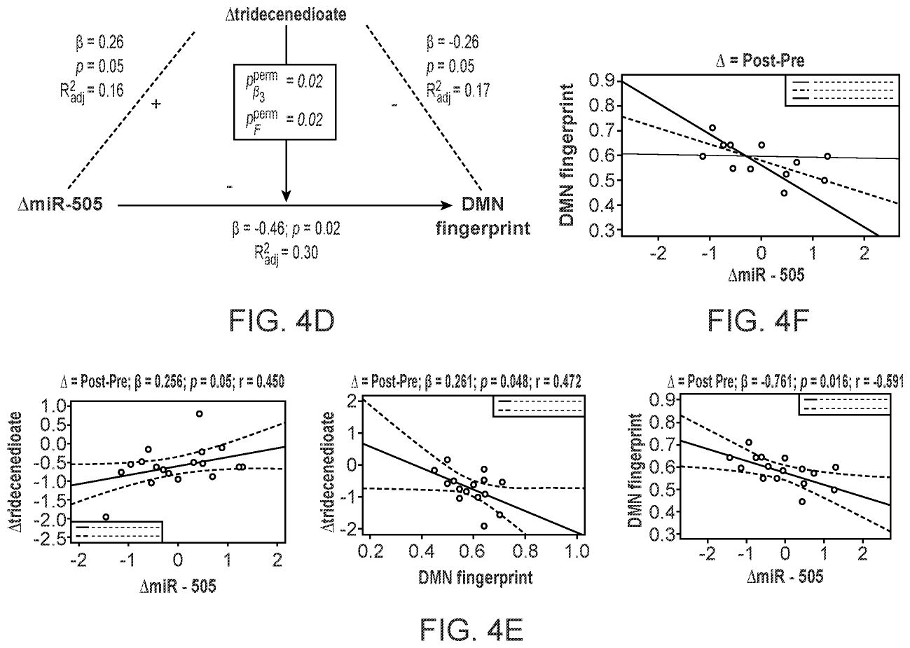

One significant moderation result is shown in FIG. 4(A-C) between ΔVR Balance (dependent variable), and DMN fingerprint, Δtridecenedioate (independent variable, moderator) with p_{F}^{perm}=0.03, p_{β}_{₃}^{perm}=0.05. The regression slope (13), p-value and adjusted R² (R_{adj}²) depicted on each arm of the triangle corresponds to the pairwise interaction results, after Cook's distance outliers removed, between those two variables with p≤0.05. FIG. 4(B) shows the pairwise interactions between the three variables ΔVR Balance, DMN fingerprint and Δtridecenedioate. There was a negative relationship between DMN fingerprint and Δtridecenedioate, a negative relationship between Δtridecenedioate and ΔVR Balance and a positive relationship between DMN fingerprint and ΔVR Balance. FIG. 4(C) plots the relationship between DMN fingerprint and ΔVR Balance with Δtridecenedioate as the moderator. Three lines corresponds to low (mean−standard deviation), medium (mean) and high (mean+standard deviation) Δtridecenedioate values.

A second significant moderation result is shown in FIG. 4(D-F) between DMN fingerprint (dependent variable), and ΔmiR-505, Δtridecenedioate (independent variable, moderator) with p_{F}^{perm}=0.02, p_{β}_{₃}^{perm}=0.02. The regression slope (β), p-value and adjusted R² (R_{adj}²) depicted on each arm of the triangle corresponds to the pairwise interaction results, after Cook's distance outliers removed, between those two variables with p≤0.05. FIG. 4(E) plots the pairwise interactions between these three variables. There was a positive relationship between ΔmiR-505 and Δtridecenedioate, a negative relationship between DMN fingerprint and Δtridecenedioate and a negative relationship between DMN fingerprint and ΔmiR-505. FIG. 4(F) depicts the relationship between Δ miR-505 and DMN fingerprint with Δtridecenedioate as the moderator. Three lines corresponds to low (mean−standard deviation), medium (mean) and high (mean+standard deviation) Δtridecenedioate values.

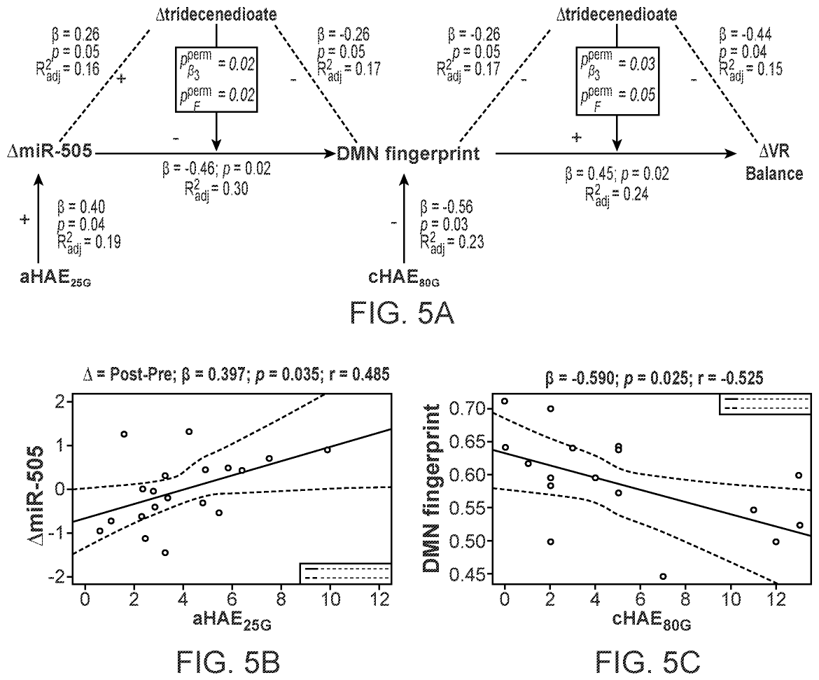

Given the same metabolomic measure moderated both results, FIG. 5A-5C integrates the two moderation analyses results along with HAE metrics. FIG. 5(A) shows that ΔVR Balance was the dependent variable and DMN fingerprint acted as the independent variable for one moderation and dependent variable for the other. Δtridecenedioate acted as the moderator for the two moderation analyses. In addition, there was a positive relationship between ΔmiR-505 and aHAE_{25G} as depicted in FIG. 5(B). FIG. 5(C) shows the negative relationship between DMN fingerprint and cHAE_{80G}.

Summary of HAE interactions: Two methods were used to assess HAE interactions: 1) extrapolation of subject-specific HAEs to predict preseason VR scores, miRNA levels, and metabolite levels (note: this analysis is purely exploratory and should only be considered for future analyses), 2) assessment of how HAEs may impact across-season changes in VR scores, miRNA, and metabolites.

At preseason, there were six significant interactions between HAEs and miRNA (5/6 related to HAEs above 80 G) and two interactions between HAEs and metabolites (2/2 related to HAEs above 25 G) (Table 4). Across-season, there were two significant interactions between HAEs and miRNAs (both positive; +β), as well as four interactions between HAEs and metabolites (all positive; +β) (Table 4). Interactions with miRNA were related to normalized HAEs (i.e. aHAE_{25G} and aHAE_{80G}). Interactions with metabolites (sebacate and suberate) were related to HAEs exceeding 25 G (cHAE_{25G} and aHAE_{25G}). Significant HAE regressions were plotted in Table 3 and their interaction terms with significant across-season mediations can be seen in FIG. 3A-3H.

TABLE 4
Across-season interactions with head acceleration events (HAEs). Four postseason
HAE metrics (cHAE_{25G}, cHAE_{80G}, aHAE_{25G}, and aHAE8_{0G}) were regressed against
mediation-related VR scores, miRNA levels, and metabolite levels. Cook's outliers
were removed prior to analyses. Results were presented up-value <0.05. Adjusted R²
(R_{adj.}²) and standardized beta coefficients (Std. β) were reported for each significant
regression.

| Session | DV | IV (HAE) | R_{adj}² | Std. β | p-value | Cook's outliers |
|---|---|---|---|---|---|---|
| Pre | 505 | cHAE_{80G} | 0.354 | −0.623 | 0.003 | 1/21 |
| Pre | 505 | cHAE_{25G} | 0.192 | −0.535 | 0.035 | 2/21 |
| Pre | 505 | aHAE_{80G} | 0.255 | −0.850 | 0.016 | 2/21 |
| Pre | 92a | cHAE_{80G} | 0.382 | −0.644 | 0.002 | 2/22 |
| Pre | 92a | aHAE_{80G} | 0.233 | −0.518 | 0.021 | 3/22 |
| Pre | 93p | cHAE_{80G} | 0.266 | −0.550 | 0.010 | 1/22 |
| Pre | undecanedioate | cHAE_{25G} | 0.144 | 0.430 | 0.046 | 1/23 |
| Pre | undecanedioate | aHAE_{25G} | 0.189 | 0.477 | 0.025 | 1/23 |
| Post-Pre | Δ505 | aHAE_{25G} | 0.191 | 0.486 | 0.035 | 1/20 |
| Post-Pre | Δ92a | aHAE_{80G} | 0.177 | 0.475 | 0.046 | 2/20 |
| Post-Pre | Δsebacate | cHAE_{25G} | 0.279 | 0.559 | 0.007 | 1/23 |
| Post-Pre | Δsebacate | aHAE_{25G} | 0.190 | 0.480 | 0.028 | 2/23 |
| Post-Pre | Δsuberate | cHAE_{25G} | 0.174 | 0.464 | 0.034 | 2/23 |
| Post-Pre | Δsuberate | aHAE_{25G} | 0.178 | 0.468 | 0.032 | 2/23 |

Additional embodiments include the following numbered embodiments:
1. A method of treating head injury, diagnosing head injury, prognosing head injury, assessing susceptibility for head injury, assessing resilience against head injury, and/or determining treatment for head injury, in a subject comprising:
   comparing, using a computer processor, at least three measures selected from the group consisting of:
a) fatty acids and metabolites thereof;
b) TCA metabolites;
c) nucleosides, nucleic acids, and derivatives thereof;
d) behavioral scores; and
e) imaging scores,
from the subject, to the same measures in a control subject.
2. A method of identifying a biological target for a therapeutic agent to treat or prevent head injury, the method comprising comparing, using a computer processor, at least three measures selected from the group consisting of:
   a) fatty acids and metabolites thereof;
   b) TCA metabolites;
   c) nucleosides, nucleic acids, and derivatives thereof;
   d) behavioral scores; and
   e) imaging scores,
from a subject, to the same measures in a control subject.
3. The method of embodiment 1 or 2, wherein the comparing comprises assessing if each of the at least three measures selected from a)-e) is increased or decreased relative to the same measure in the control subject.
4. The method any one of embodiments 1-3, wherein the comparing further comprises defining a positive or negative relationship between any two of the at least three measures from a)-e), in the subject,
   wherein a positive relationship comprises both of the two measures being increased or both of the two measures being decreased relative to the same two measures in the control subject, and
   a negative relationship comprises one of the two measures being increased and the other of the two measures being decreased, wherein the increase or decrease is relative to the same measure in the control subject.
5. The method of embodiment 4, wherein all three possible relationships between any two of the at least three measures of a)-e) are defined, to establish a three-way association between the at least three measures selected from a)-e).
6. The method of embodiment 5, further comprising analyzing the three-way association via moderation analysis.
7. The method of any one of embodiments 1-6, further comprising calculating using the computer processor and the three-way association, a diagnostic likelihood of diagnosing head injury, prognosing head injury, assessing susceptibility for head injury, assessing resilience against head injury, and/or determining treatment for head injury, in the subject.
8. The method of embodiment 7, wherein the calculating further comprises integrating peripheral protein measurements in the subject and the control subject into the calculation of diagnostic likelihood.
9. The method of embodiment 7 or 8, wherein the calculating further comprises integrating peripheral protein measurements in the subject and the control subject into the calculation of diagnostic likelihood.
10. The method of embodiment 9, wherein the peripheral protein is selected from the group consisting of IL-β, TNFα, IL-6, UCH-L1, tau, NfL, and GFAP.
11. The method of any one of embodiments 7-10, wherein the calculating further comprises integrating hormone measures in the subject and the control subject into the calculation of diagnostic likelihood.
12. The method of embodiment 11, wherein the hormone comprises one or more hormones selected from the group consisting of progesterone, estrogen, testosterone, androsterone glucuronide; etiocholanolone glucuronide; 5α-androstan-3α,17α-diol monosulfate; androstenediol (3β, 17α) monosulfate; androstenediol (3α, 17β) disulfate; androstenediol (3α, 17α) monosulfate; 5α-androstan-3α,17β-diol monosulfate; 5α-androstan-3α,17β-diol disulfate; 5α-androstan-3α,17β-diol monosulfate; pregnenolone steroids, sterols, corticosteroids.
13. The method of any one of embodiments 7-12, wherein the calculating further comprises integrating genotype data for the subject and the control subject into the calculation of diagnostic likelihood.
14. The method of embodiment 13, wherein the genotype data comprises SNP at a gene such selected from the group consisting of DARC, TPH2, or KIAA0319.
15. The method of any one of embodiments 1-14, wherein prognosing comprises prognosing the longitudinal course of the head injury.
16. The method of any one of embodiments 1-15, further comprising integrating the three way association with genotype data, hormone measurements, and peripheral protein measurements in the subject and control subject.
17. The method of any one of embodiments 1-16, wherein the fatty acids and metabolites thereof comprise one or more of tridecenedioate, 2-hydroxyglutarate, undecanedioate, sebacate, suberate, and heptanoate, 7-hydroxyoctanoate, octanoylcarnitine, decanolycarnitine, cis-4-decenoylcarnitine, adipoylcarnitine, suberoylcarnitine; glutarate, 3-hydroxyadipate, pimelate, heptenedioate, azelate, dodecadienoate, hexadecendioate, 2-hydroxysebacate, caproate, caprylate, pelargonate, caprate, undecanoate, 10-undecanoate, palmitoleate, stearidonate, hexadecadienoate, linoleate (18:2n6), linoleate (18:3n3 or 6), propinoylcarnitine, 3-hydroxybutyroylglycine, stearoylcarnitine, undecenoylcarnitine, linolenoylcarnitine, deoxycarnitine, palmitoylcholine, oleoylcholine, palmitoleoylcholine, dihomo-linoeoyl-choline, stearoylcholine, docosahexaenoylcholine, arachidonolycholine, 2S-3R-dihydroxybutyrate, 3,4-dihydroxyglutarate, N-palmitoylserine, cortisol, cortoloneglucuronide, 1-carboxyethylphenylalanine, cortisone, paraxanthine, 1,7-dimethylurate, 1-methlxanthine, 1,2,3-benzentriol sulfate, 5-acetylamino-6-formylamino-3-methyluracil, O-sulfo-L-tyrosine, chiro-inositol, choline, choline phosphate, phosphoethanolamine, phosphatidylethanolamines, phosphatidylinositols, lysophospholipids, plasmalogens, glycerolipid metabolites, sphingnolipids, diacylglycerols, ceramides, hexosylceramides, sphingnosines, or a conjugate acid thereof
18. The method of any one of embodiments 1-17, wherein the nucleosides, nucleic acids, and derivatives thereof comprises one or more of miRNA, RNA, mRNA, DNA, tRNA, rRNA and sRNA.
19. The method of embodiment 18, wherein nucleosides, nucleic acids, and derivatives thereof comprise miRNA comprising one or more of miR-20a, miR-505, miR-3623p, miR-30d, miR-92a, miR-486, miR-92a, miR-93p, and miR-151-5p.
20. The method of any one of embodiments 1-19, wherein the TCA metabolites comprise one or more of adenine, nicotinamide, adenosine, acetyl-CoA, citrate, aconitate, isocitrate, NAD+, NADH, CO₂, O₂, α-ketoglutarate, L-2-hydroxyglutrate, D-2-hydroxyglutrate, GTP, GDP, ATP, ADP, phosphate, pyrophosphate, S-CoA, coenzyme A (CoA), succinate, FAD, FADH₂, fumarate, malate, oxaloacetate, glucose, pyruvate, lactate, glycerate, ribose and conjugate acids any thereof.
21. The method of any one of embodiments 1-20, wherein the behavioral score comprises one or more of virtual reality testing (VR), Post Concussion Symptom Scale (PCSS), Glasgow Coma Scale (GCS), eye tracking, N-back test, reaction time to a stimulus, ear opening and eye opening, surface righting, air righting, forelimb grasp, auditory startle, negative geotaxis, openfield traversal, cliff aversion, Barnes maze, elevated plus maze, Jamar dynamometer, handheld dynamometry, manual muscle testing (MMT), isokinetic dynamometry, trunk stability test (TST), unilateral hip bridge endurance test (UHBE), pronator sign, Barré sign, Romberg test, Landau reflex, particle suspension, sensory reflex (pinprick, light touch, position, vibration, and charger), reflex (biceps, triceps, brachioradialis, patellar, and ankle), Moro reflex, tonic neck response, sucking reflex, palmer and planter grasp reflex, parachute response, neck on body righting reaction (NOB), body on body righting reaction (BOB), ear opening auditory reflex, static compliance, physical volume of ear canal, contralateral reflex, ipsilateral reflex, tympanometry, Y-maze, Novel Object Recognition Task, STPI (State-Trait Personality Inventory), the Five Dimensional Curiosity Scale, Self-Curiosity Attitude Interests Scale, Curiosity and Exploration Inventory-II, State-Trait Personality Inventory (STPI), subscales of the Sensation Seeking Scale (SSS), Bayley Scales of Infant Development (BSID-III) (1-42 months), the Mullen Scales of Early Learning (1-68 months), the Fagan Test of Infant Intelligence (FTII) (Birth-12 months), Griffith's Mental Development Scales I (0-2 years), Battelle Developmental Inventory (BDI) (Birth-8 years), and the Vineland Adaptive Behavior Scale (0-18 years) or cognition tests including ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, and a computerized tested selected from Cantab Mobile, Cognigram, Cognivue, Cognision, or Automated Neuropsychological Assessment Metrics Cognitive Performance Test (CPT).
22. The method of any one of embodiments 1-21 wherein the imaging score is attained by a method comprising one or more of magnetic resonance imaging (MRI), Positron Emission Tomography (PET), computerized tomography (CT), cranial ultrasound, diffuse optical imaging (DOI), magnetoencephalography (MEG), and Electroencephalography (EEG).
23. The method of embodiment 22, wherein the imaging is of one or more of brain systems selected from Visual (VIS), Somato-Motor (SM), Dorsal Attention (DA), Ventral Attention (VA), Limbic System (L), Fronto-Parietal (FP), Default Mode Network (DMN) and subcortical regions (SUBC).
24. The method of any one of embodiments 2-23, wherein the biological targets comprise enzymes or compounds in one or more of the fatty acid pathway, membrane synthesis, TCA, mitochondrial energy metabolism pathway, oxidative phosphorylation, glycolysis, gluconeogenesis, pyruvate metabolism, electron transport chain, peptide synthesis and metabolism, and amino acid metabolism.
25. The method of any one of embodiments 2-24, further comprising selecting a lead compound targeting the biological target and assessing changes in three-way associations, diagnostic likelihoods after administration of the lead compound to the subject, relative to those measures in a second control subject recovered or recovering from a head injury.
26. The method of any one of embodiments 1-25, wherein the control subject is a healthy subject.
27. The method of any one of embodiments 1-26, wherein the control subject has a head injury.
28. The method of any one of embodiments 1-27, wherein the head injury comprises concussion.
29. The method of any one of embodiments 1-28, wherein measures a)-b) are measured by contacting a biological sample from the subject with an agent specific for any one of a)-c) and detecting an interaction between the agent and the any one of a)-c).
30. A method for detecting at least two biomarkers of head injury in a subject, the biomarkers selected from the group consisting of:
   a) fatty acids and metabolites thereof;
   b) TCA metabolites; and
   c) nucleosides, nucleic acids, and derivatives thereof,
      the method comprising contacting a biological sample from the subject with an agent specific for the biomarker(s) and detecting the presence or absence of the biomarker in the biological sample based on the agent's interaction with the biomarker to determine a head injury biomarker profile for the subject and comparing the subject's head injury biomarker profile to a control head injury biomarker profile.
31. The method of any one of embodiments 1-30 wherein reactive oxygen species (ROS) and free radical species implicated in brain injury are involved such as superoxide, hydrogen peroxide, acrolein, 4-hydroxynenal, and nitric oxide.
32. The method of any of one embodiments 1-31, wherein mitochondrial dysfunction underlies the neurological pathophysiology of any disease of interest including, but not limited to, Alzheimer's Disease, Parkinson's Disease, neurodegenerative disease, stroke, and epilepsy.
33. The method of any of one embodiments 1-31, wherein mitochondrial dysfunction underlies neuropsychiatric disorders, including, but not limited to, depression, schizophrenia, and bipolar disorder.
34. The method of any of one embodiments 1-31, wherein mitochondrial dysfunction underlies disorders related to addiction, including, but not limited to, substance abuse disorders, alcoholism, and gambling.

### REFERENCES

1. Gan Z S, Stein S C, Swanson R, Guan S, Garcia L, Mehta D, et al. Blood biomarkers for traumatic brain injury: A quantitative assessment of diagnostic and prognostic accuracy. Front Neurol. 2019; 10.
2. Papa L, Edwards D, Ramia M. Exploring Serum Biomarkers for Mild Traumatic Brain Injury. In: Kobeissy F H, editor. Brain Neurotrauma Mol. Neuropsychol. Rehabil. Asp., Boca Raton (Fla.): CRC Press/Taylor & Francis; 2015.
3. Berger R P, Beers S R, Richichi R, Wiesman D, Adelson P D. Serum biomarker concentrations and outcome after pediatric traumatic brain injury. J Neurotrauma. 2007; 24:1793-1801.
4. Shahim P, Tegner Y, Wilson D H, Randall J, Skillbäck T, Pazooki D, et al. Blood biomarkers for brain injury in concussed professional ice hockey players. JAMA Neurol. 2014; 71.
5. Bigler E D. Neuroimaging biomarkers in mild traumatic brain injury (mTBI). Neuropsychol Rev. 2013; 23:169-209.
6. Dash P K, Zhao J, Hergenroeder G, Moore A N. Biomarkers for the Diagnosis, Prognosis, and Evaluation of Treatment Efficacy for Traumatic Brain Injury. Neurotherapeutics. 2010; 7:100-114.
7. Agoston D V, Shutes-David A, Peskind E R. Brain Injury Biofluid biomarkers of traumatic brain injury. Brain Inj. 2017; 31:1195-1203.
8. Orešič M, Posti J P, Kamstrup-Nielsen M H, Takala R S K, Lingsma H F, Mattila I, et al. Human Serum Metabolites Associate With Severity and Patient Outcomes in Traumatic Brain Injury. EBioMedicine. 2016; 12:118-126.
9. Berger R P. The Use of Serum Biomarkers to Predict Outcome After Traumatic Brain Injury in Adults and Children. J Head Trauma Rehabil. 2006; 21:315-333.
10. Guingab-Cagmat J D, Cagmat E B, Hayes R L, Anagli J. Integration of proteomics, bioinformatics, and systems biology in traumatic brain injury biomarker discovery. Front Neurol. 2013; 4.
11. Bigler E D, Farrer T J, Pertab J L, James K, Petrie J A, Hedges D W. Reaffirmed limitations of meta-analytic methods in the study of mild traumatic brain injury: A response to Rohling et al. Clin Neuropsychol. 2013; 27:176-214.
12. Nizamutdinov D, Shapiro L A. Overview of Traumatic Brain Injury: An Immunological Context. Brain Sci. 2017; 7.
13. Shao C, Roberts K N, Markesbery W R, Scheff S W, Lovell M A. Oxidative stress in head trauma in aging. Free Radic Biol Med. 2006; 41:77-85.
14. Blennow K, Hardy J, Zetterberg H. The Neuropathology and Neurobiology of Traumatic Brain Injury. Neuron. 2012; 76:886-899.
15. Elder G A, Gama Sosa M A, De Gasperi R, Stone J R, Dickstein D L, Haghighi F, et al. Vascular and inflammatory factors in the pathophysiology of blast-induced brain injury. Front Neurol. 2015; 6:1-22.
16. Woodcock T, Morganti-Kossmann M C. The role of markers of inflammation in traumatic brain injury. Front Neurol. 2013; 4:1-18.
17. Chiu C-C, Liao Y-E, Yang L-Y, Wang J-Y, Tweedie D, Karnati H K, et al. Neuroinflammation in animal models of traumatic brain injury. J Neurosci Methods. 2016; 272:38-49.
18. Signoretti S, Lazzarino G, Tavazzi B, Vagnozzi R. The pathophysiology of concussion. P M R J Inj Funct Rehabil. 2011; 3: S359-368.
19. Signoretti S, Vagnozzi R, Tavazza B, Lazzarino G. Biochemical and neurochemical sequelae following mild traumatic brain injury: summary of experimental data and clinical implications. J Neurosurg. 2010; 29:E1.
20. Shao C. Oxidative stress and mitochondrial dysfunction in traumatic brain injury in aging. Univ Kentucky Dr Diss. 2007; 533.
21. Hiebert J B, Shen Q, Thimmesch A R, Pierce J D. Traumatic Brain Injury and Mitochondrial Dysfunction. Am J Med Sci. 2015; 350:132-138.
22. Vagnozzi R, Marmarou A, Tavazzi B, Signoretti S, Di Pierro D, Del Bolgia F D, et al. Changes of cerebral energy metabolism and lipid peroxidation in rats leading to mitochondrial dysfunction after diffuse brain injury. J Neurotrauma. 1999; 16:903-913.
23. Verweij B H, Muizelaar P, Vinas F C, Peterson P L, Xiong Y, Lee C P. Impaired cerebral mitochondrial function after traumatic brain injury in humans. J Neurosurg. 2000; 93:815-820.
24. Gonzalez-Dominguez R. Medium-chain Fatty Acids as Biomarkers of Mitochondrial Dysfunction in Traumatic Brain Injury. EBioMedicine. 2016; 12:8-9.
25. Robertson C L, Saraswati M, Fiskum G. Mitochondrial dysfunction early after traumatic brain injury in immature rats. J Neurochem. 2007; 101:1248-1257.
26. Xiong Y, Gu Q, Peterson P L, Muizelaar J P, Lee C P. Mitochondrial dysfunction and calcium perturbation induced by traumatic brain injury. J Neurotrauma. 1997; 14:23-34.
27. Siekevitz P. Powerhouse of the cell. Sci Am. 1957; 197:131-144.
28. Wisnovsky S, Lei E K, Jean S R, Kelley S O. Mitochondrial Chemical Biology: New Probes Elucidate the Secrets of the Powerhouse of the Cell. Cell Chem Biol. 2016; 23:917-927.
29. Wanders R J A, Ruiter J P N, Ijlst L, Waterham H R, Houten S M. The enzymology of mitochondrial fatty acid beta-oxidation and its application to follow-up analysis of positive neonatal screening results. J Inherit Metab Dis. 2010; 33:479-494.
30. Kunau W H, Dommes V, Schulz H. β-Oxidation of fatty acids in mitochondria, peroxisomes, and bacteria: A century of continued progress. Prog Lipid Res. 1995; 34:267-342.
31. Schulz H. Beta oxidation of fatty acids. Biochim Biophys Acta (BBA)/Lipids Lipid Metab. 1991; 1081:109-120.
32. Titorenko V I, Terlecky S R. Peroxisome Metabolism and Cellular Aging. Traffic. 2011; 12:252-259.
33. Wanders R J A, Waterham H R, Ferdinandusse S. Metabolic interplay between peroxisomes and other subcellular organelles including mitochondria and the endoplasmic reticulum. Front Cell Dev Biol. 2016; 3.
34. Lodhi I J, Semenkovich C F. Peroxisomes: A nexus for lipid metabolism and cellular signaling. Cell Metab. 2014; 19:380-392.
35. Eaton S, Bartlett K, Pourfarzam M. Mammalian mitochondrial β-oxidation. Biochem J. 1996; 320:345-357.
36. Bartlett K, Eaton S. Mitochondrial β-oxidation. Eur J Biochem. 2004; 271:462-469.
37. Fernie A R, Carrari F, Sweetlove L J. Respiratory metabolism: Glycolysis, the TCA cycle and mitochondrial electron transport. Curr Opin Plant Biol. 2004; 7:254-261.
38. Bhatti J S, Bhatti G K, Reddy P H. Mitochondrial dysfunction and oxidative stress in metabolic disorders—A step towards mitochondria based therapeutic strategies. Biochim Biophys Acta-Mol Basis Dis. 2017; 1863:1066-1077.
39. Bhat A H, Dar K B, Anees S, Zargar M A, Masood A, Sofi M A, et al. Oxidative stress, mitochondrial dysfunction and neurodegenerative diseases; a mechanistic insight. Biomed Pharmacother. 2015; 74:101-110.
40. Wang C H, Wu S B, Wu Y T, Wei Y H. Oxidative stress response elicited by mitochondrial dysfunction: Implication in the pathophysiology of aging. Exp Biol Med. 2013; 238:450-460.
41. Ježek J, Cooper K F, Strich R. Reactive oxygen species and mitochondrial dynamics: The yin and yang of mitochondrial dysfunction and cancer progression. Antioxidants. 2018; 7.
42. Sports-related concussions in youth: Improving the science, changing the culture. National Academies Press, Washington, DC; 2014.
43. Moran L M, Taylor H G, Rusin J, Bangert B, Dietrich A, Nuss K E, et al. Do postconcussive symptoms discriminate injury severity in pediatric mild traumatic brain injury? J Head Trauma Rehabil. 2011; 26:348-354.
44. Bari S, Svaldi D O, Jang I, Shenk T E, Poole V N, Lee T, et al. Dependence on subconcussive impacts of brain metabolism in collision sport athletes: an MR spectroscopic study. Brain Imaging Behav. 2019; 13:735-749.
45. Abbas K. Effects of concussive and repetitive subconcussive injury in high school football athletes using resting state FMRI. Purdue University, 2016.
46. Talavage T_{M}, Nauman E A, Breedlove E L, Yoruk U, Dye A E, Morigaki K E, et al. Functionally-Detected Cognitive Impairment in High School Football Players without Clinically-Diagnosed Concussion. J Neurotrauma. 2014; 31:327-338.
47. Talavage T_{M}, Nauman E A, Leverenz L J. The role of medical imaging in the recharacterization of mild traumatic brain injury using youth sports as a laboratory. Front Neurol. 2016; 6.
48. Abbas K, Shenk T E, Poole V N, Breedlove E L, Leverenz L J, Nauman E A, et al. Alteration of Default Mode Network in High School Football Athletes Due to Repetitive Subconcussive Mild Traumatic Brain Injury: A Resting-State Functional Magnetic Resonance Imaging Study. Brain Connect. 2015; 5:91-101.
49. Svaldi D O, Joshi C, Robinson M E, Shenk T E, Abbas K, Nauman E A, et al. Cerebrovascular Reactivity Alterations in Asymptomatic High School Football Players. Dev Neuropsychol. 2015; 40:80-84.
50. Bailes J E, Petraglia A L, Omalu B I, Nauman E, Talavage T. Role of subconcussion in repetitive mild traumatic brain injury. J Neurosurg. 2013; 119:1235-1245.
51. Slobounov S M, Walter A, Breiter H C, Zhu D C, Bai X X, Bream T, et al. The effect of repetitive subconcussive collisions on brain integrity in collegiate football players over a single football season: A multi-modal neuroimaging study. Neuroimage-Clinical. 2017; 14:708-718.
52. Chen Y, Herrold A A, Martinovich Z, Blood A J, Vike N, Walter A E, et al. Brain perfusion mediates the relationship between miRNA levels and postural control. 2019.5 Dec. 2019.
53. Teel E, Gay M, Johnson B, Slobounov S. Determining sensitivity/specificity of virtual reality-based neuropsychological tool for detecting residual abnormalities following sport-related concussion. Neuropsychology. 2016; 30:474-483.
54. Teel E F, Slobounov S M. Validation of a virtual reality balance module for use in clinical concussion assessment and management. Clin J Sport Med Off J Can Acad Sport Med. 2015; 25:144-148.
55. Luria A R. Recovery of brain functions after military traumasMoscow: Moscow State University Publisher; 1948.
56. Luria A R. Higher cortical functions and its deficits after local brain traumasMoscow: Moscow University Publisher; 1962.
57. Luria A R. Foundation of Neuropsychology.Moscow: Moscow University Publisher; 1973.
58. Cox R W. AFNI: software for analysis and visualization of functional magnetic resonance neuroimages. Comput Biomed Res. 1996; 29:162-173.
59. Jenkinson M, Beckmann C F, Behrens T E J, Woolrich M W, Smith S M. FSL. Neuroimage. 2012; 62:782-790.
60. Smith S M, Jenkinson M, Woolrich M W, Beckmann C F, Behrens T E J, Johansen-Berg H, et al. Advances in functional and structural MR image analysis and implementation as FSL. Neuroimage, vol. 23, Neuroimage; 2004.
61. Bari S, Amico E, Vike N, Talavage T_{M}, Go˜Ni J. Uncovering multi-site identifiability based on resting-state functional connectomes. 2019.2019.
62. Shen X, Tokoglu F, Papademetris X, Constable R T. Groupwise whole-brain parcellation from resting-state fMRI data for network node identification. Neuroimage. 2013; 82:403-415.
63. Thomas Yeo B T, Krienen F M, Sepulcre J, Sabuncu M R, Lashkari D, Hollinshead M, et al. The organization of the human cerebral cortex estimated by intrinsic functional connectivity. J Neurophysiol. 2011; 106:1125-1165.
64. Amico E, Marinazzo D, Di Perri C, Heine L, Annen J, Martial C, et al. Mapping the functional connectome traits of levels of consciousness. Neuroimage. 2017; 148:201-211.
65. Amico E, Goñi J. The quest for identifiability in human functional connectomes. Sci Rep. 2018; 8:1-14.
66. Finn E S, Shen X, Scheinost D, Rosenberg M D, Huang J, Chun M M, et al. Functional connectome fingerprinting: Identifying individuals using patterns of brain connectivity. Nat Neurosci. 2015; 18:1664-1671.
67. Team R Core. A Language and Environment for Statistical Computing. 2013.
68. Breiman L. Random forests. Mach Learn. 2001; 45:5-32.
69. Papa L, Slobounov S M, Breiter H C, Walter A, Bream T, Seidenberg P, et al. Elevations in MicroRNA Biomarkers in Serum Are Associated with Measures of Concussion, Neurocognitive Function, and Subconcussive Trauma over a Single National Collegiate Athletic Association Division I Season in Collegiate Football Players. J Neurotrauma. 2019; 36:1343-1351.
70. Cook R D. Detection of influential observations in linear regression. Technometrics. 1977; 22:494-508.
71. Cook R D. Influential observations in linear regression. J Am Stat Assoc. 1979; 74:169-174.
72. Altman N, Krzywinski M. Points of Significance: Analyzing outliers: Influential or nuisance? Nat Methods. 2016; 13:281-282.
73. McCuen E, Svaldi D, Breedlove K, Kraz N, Cummiskey B, Breedlove E L, et al. Collegiate women's soccer players suffer greater cumulative head impacts than their high school counterparts. J Biomech. 2015; 48:3720-3723.
74. Broglio S P, Schnebel B, Sosnoff J J, Shin S, Fend X, He X, et al. Biomechanical properties of concussions in high school football. Med Sci Sports Exerc. 2010; 42:2064-2071.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety. Further embodiments are set forth in the following claims.

What is claimed is:

1. A method of treating head injury, diagnosing head injury, prognosing head injury, assessing susceptibility for head injury, assessing resilience against head injury, and/or determining treatment for head injury, in a subject comprising:
comparing, using a computer processor, at least three measures selected from the group consisting of:
a) fatty acids and metabolites thereof;
b) TCA metabolites;
c) nucleosides, nucleic acids, and derivatives thereof;
d) behavioral scores; and
e) imaging scores,
from the subject, to the same measures in a control subject.

2. A method of identifying a biological target for a therapeutic agent to treat or prevent head injury, the method comprising comparing, using a computer processor, at least three measures selected from the group consisting of:
f) fatty acids and metabolites thereof;
g) TCA metabolites;
h) nucleosides, nucleic acids, and derivatives thereof;
i) behavioral scores; and
j) imaging scores,
from a subject, to the same measures in a control subject.

3. The method of claim 1, wherein the comparing comprises assessing if each of the at least three measures selected from a)-e) is increased or decreased relative to the same measure in the control subject.

4. The method of claim 1, wherein the comparing further comprises defining a positive or negative relationship between any two of the at least three measures from a)-e), in the subject,
wherein a positive relationship comprises both of the two measures being increased or both of the two measures being decreased relative to the same two measures in the control subject, and
a negative relationship comprises one of the two measures being increased and the other of the two measures being decreased, wherein the increase or decrease is relative to the same measure in the control subject.

5. The method of claim 4, wherein all three possible relationships between any two of the at least three measures of a)-e) are defined, to establish a three-way association between the at least three measures selected from a)-e).

6. The method of claim 5, further comprising analyzing the three-way association via moderation analysis.

7. The method of claim 1, further comprising calculating using the computer processor and the three-way association, a diagnostic likelihood of diagnosing head injury, prognosing head injury, assessing susceptibility for head injury, assessing resilience against head injury, and/or determining treatment for head injury, in the subject.

8. The method of claim 7, wherein the calculating further comprises integrating peripheral protein measurements in the subject and the control subject into the calculation of diagnostic likelihood.

9. The method of claim 7, wherein the calculating further comprises integrating peripheral protein measurements in the subject and the control subject into the calculation of diagnostic likelihood.

10. The method of claim 9, wherein the peripheral protein is selected from the group consisting of IL-1β, TNFα, IL-6, UCH-L1, tau, NfL, and GFAP.

11. The method of claim 7, wherein the calculating further comprises integrating hormone measures in the subject and the control subject into the calculation of diagnostic likelihood.

12. The method of claim 11, wherein the hormone comprises one or more hormones selected from the group consisting of progesterone, estrogen, testosterone, androsterone glucuronide; etiocholanolone glucuronide; 5α-androstan-3α,17α-diol monosulfate; androstenediol (3β, 17α) monosulfate; androstenediol (3α, 17β) disulfate; androstenediol (3α, 17α) monosulfate; 5α-androstan-3α,17β-diol monosulfate; 5α-androstan-3α,17β-diol disulfate; 5α-androstan-3β,17β-diol monosulfate; pregnenolone steroids, sterols, corticosteroids.

13. The method of claim 7, wherein the calculating further comprises integrating genotype data for the subject and the control subject into the calculation of diagnostic likelihood.

14. The method of claim 13, wherein the genotype data comprises SNP at a gene such selected from the group consisting of DARC, TPH2, or KIAA0319.

15. The method of claim 1, wherein prognosing comprises prognosing the longitudinal course of the head injury.

16. The method of claim 1, further comprising integrating the three way association with genotype data, hormone measurements, and peripheral protein measurements in the subject and control subject.

17. The method of claim 1, wherein the fatty acids and metabolites thereof comprise one or more of tridecenedioate, 2-hydroxyglutarate, undecanedioate, sebacate, suberate, and heptanoate, 7-hydroxyoctanoate, octanoylcarnitine, decanolycarnitine, cis-4-decenoylcarnitine, adipoylcarnitine, suberoylcarnitine; glutarate, 3-hydroxyadipate, pimelate, heptenedioate, azelate, dodecadienoate, hexadecendioate, 2-hydroxysebacate, caproate, caprylate, pelargonate, caprate, undecanoate, 10-undecanoate, palmitoleate, stearidonate, hexadecadienoate, linoleate (18:2n6), linoleate (18:3n3 or 6), propinoylcarnitine, 3-hydroxybutyroylglycine, stearoylcarnitine, undecenoylcarnitine, linolenoylcarnitine, deoxycarnitine, palmitoylcholine, oleoylcholine, palmitoleoylcholine, dihomo-linoeoyl-choline, stearoylcholine, docosahexaenoylcholine, arachidonolycholine, 2S-3R-dihydroxybutyrate, 3,4-dihydroxyglutarate, N-palmitoylserine, cortisol, cortoloneglucuronide, 1-carboxyethylphenylalanine, cortisone, paraxanthine, 1,7-dimethylurate, 1-methlxanthine, 1,2,3-benzentriol sulfate, 5-acetylamino-6-formylamino-3-methyluracil, O-sulfo-L-tyrosine, chiro-inositol, choline, choline phosphate, phosphoethanolamine, phosphatidylethanolamines, phosphatidylinositols, lysophospholipids, plasmalogens, glycerolipid metabolites, sphingnolipids, diacylglycerols, ceramides, hexosylceramides, sphingnosines, or a conjugate acid thereof.

18. The method of claim 1, wherein the nucleosides, nucleic acids, and derivatives thereof comprises one or more of miRNA, RNA, mRNA, DNA, tRNA, rRNA and sRNA.

19. The method of claim 18, wherein nucleosides, nucleic acids, and derivatives thereof comprise miRNA comprising one or more of miR-20a, miR-505, miR-3623p, miR-30d, miR-92a, miR-486, miR-92a, miR-93p, and miR-151-5p.

20. The method of claim 1, wherein the TCA metabolites comprise one or more of adenine, nicotinamide, adenosine, acetyl-CoA, citrate, aconitate, isocitrate, NAD+, NADH, CO₂, O₂, α-ketoglutarate, L-2-hydroxyglutrate, D-2-hydroxyglutrate, GTP, GDP, ATP, ADP, phosphate, pyrophosphate, S-CoA, coenzyme A (CoA), succinate, FAD, FADH₂, fumarate, malate, oxaloacetate, glucose, pyruvate, lactate, glycerate, ribose and conjugate acids any thereof.
